SEQUENCE LISTING (5 sequences)

SEQ ID NO: 1 - DNA, 2846 bp, Homo sapiens
  CDS (126)..(2375)
ctgaaaaaggatcctgactgaaagctagaggcattgaggagcctgaagattctcaggttttaaagacgctagagtgccaaagaagactttgaagtgtgaaaacatttcctgtaattgaaaccaaaatgtcatttatagatccttaccagcacattatagtggagcaccagtattcccacaagtttacggtagtggtgttacgtgccaccaaagtgacaaagggggcctttggtgacatgcttgatactccagatccctatgtggaactttttatctctacaacccctgacagcaggaagagaacaagacatttcaataatgacataaaccctgtgtggaatgagacctttgaatttattttggatcctaatcaggaaaatgttttggagattacgttaatggatgccaattatgtcatggatgaaactctagggacagcaacatttactgtatcttctatgaaggtgggagaaaagaaagaagttccttttattttcaaccaagtcactgaaatggttctagaaatgtctcttgaagtttgctcatgcccagacctacgatttagtatggctctgtgtgatcaggagaagactttcagacaacagagaaaagaacacataagggagagcatgaagaaactcttgggtccaaagaatagtgaaggattgcattctgcacgtgatgtgcctgtggtagccatattgggttcaggtgggggtttccgagccatggtgggattctctggtgtgatgaaggcattatacgaatcaggaattctggattgtgctacctacgttgctggtctttctggctccacctggtatatgtcaaccttgtattctcaccctgattttccagagaaagggccagaggagattaatgaagaactaatgaaaaatgttagccacaatccccttttacttctcacaccacagaaagttaaaagatatgttgagtctttatggaagaagaaaagctctggacaacctgtcacctttactgatatctttgggatgttaataggagaaacactaattcataatagaatgaatactactctgagcagtttgaaggaaaaagttaatactgcacaatgccctttacctcttttcacctgtcttcatgtcaaacctgacgtttcagagctgatgtttgcagattgggttgaatttagtccatacgaaattggcatggctaaatatggtacttttatggctcccgacttatttggaagcaaattttttatgggaacagtcgttaagaagtatgaagaaaaccccttgcatttcttaatgggtgtctggggcagtgccttttccatattgttcaacagagttttgggcgtttctggttcacaaagcagaggctccacaatggaggaagaattagaaaatattaccacaaagcatattgtgagtaatgatagctcggacagtgatgatgaatcacacgaacccaaaggcactgaaaatgaagatgctggaagtgactatcaaagtgataatcaagcaagttggattcatcgtatgataatggccttggtgagtgattcagctttattcaataccagagaaggacgtgctgggaaggtacacaacttcatgctgggcttgaatctcaatacatcttatccactgtctcctttgagtgactttgccacacaggactcctttgatgatgatgaactggatgcagctgtagcagatcctgatgaatttgagcgaatatatgagcctctggatgtcaaaagtaaaaagattcatgtagtggacagtgggctcacatttaacctgccgtatcccttgatactgagacctcagagaggggttgatctcataatctcctttgacttttctgcaaggccaagtgactctagtcctccgttcaaggaacttctacttgcagaaaagtgggctaaaatgaacaagctcccctttccaaagattgatccttatgtgtttgatcgggaagggctgaaggagtgctatgtctttaaacccaagaatcctgatatggagaaagattgcccaaccatcatccactttgttctggccaacatcaacttcagaaagtacaaggctccaggtgttccaagggaaactgaggaagagaaagaaatcgctgactttgatatttttgatgacccagaatcaccattttcaaccttcaattttcaatatccaaatcaagcattcaaaagactacatgatcttatgcacttcaatactctgaacaacattgatgtgataaaagaagccatggttgaaagcattgaatatagaagacagaatccatctcgttgctctgtttcccttagtaatgttgaggcaagaagatttttcaacaaggagtttctaagtaaacccaaagcatagttcatgtactggaaacggcagcagtttctgatgctgaggcagtttgcaatcccatgacaactggatttaaaagtacagtacagatagtcgtactgatcatgagagactggctgatactcaaagttgcagttacttagctgcatgagaataatactattataagttaggttgacaaatgatgttgattatgtaaggatatacttagctacattttcagtcagtatgaacttcctgatacaaatgtagggatatatactgtatttttaaacatttctcaccaactttcttatgtgtgttctttttaaaaattttttttcttttaaaatatttaacagttcaatctcaataagacctcgcattatgtatgaatgttattcactgactagatttattcataccatgagacaacactatttttatttatatatgcatatatatacatacatgaaataaatacatcaatataaaaat

SEQ ID NO: 2 - protein, 749 aa, Homo sapiens
MSFIDPYQHIIVEHQYSHKFTVVVLRATKVTKGAFGDMLDTPDPYVELFISTTPDSRKRTRHFNNDINPVWNETFEFILDPNQENVLEITLMDANYVMDETLGTATFTVSSMKVGEKKEVPFIFNQVTEMVLEMSLEVCSCPDLRFSMALCDQEKTFRQQRKEHIRESMKKLLGPKNSEGLHSARDVPVVAILGSGGGFRAMVGFSGVMKALYESGILDCATYVAGLSGSTWYMSTLYSHPDFPEKGPEEINEELMKNVSHNPLLLLTPQKVKRYVESLWKKKSSGQPVTFTDIFGMLIGETLIHNRMNTTLSSLKEKVNTAQCPLPLFTCLHVKPDVSELMFADWVEFSPYEIGMAKYGTFMAPDLFGSKFFMGTVVKKYEENPLHFLMGVWGSAFSILFNRVLGVSGSQSRGSTMEEELENITTKHIVSNDSSDSDDESHEPKGTENEDAGSDYQSDNQASWIHRMIMALVSDSALFNTREGRAGKVHNFMLGLNLNTSYPLSPLSDFATQDSFDDDELDAAVADPDEFERIYEPLDVKSKKIHVVDSGLTFNLPYPLILRPQRGVDLIISFDFSARPSDSSPPFKELLLAEKWAKMNKLPFPKIDPYVFDREGLKECYVFKPKNPDMEKDCPTIIHFVLANINFRKYKAPGVPRETEEEKEIADFDIFDDPESPFSTFNFQYPNQAFKRLHDLMHFNTLNNIDVIKEAMVESIEYRRQNPSRCSVSLSNVEARRFFNKEFLSKPKA

SEQ ID NO: 3 - DNA, 2787 bp, Mus musculus
  CDS (109)..(2355)
ggcacagagaagcctgaggattctcatttaactctgggaactgcttcaagaagctacagtaccatagaagacctgggaagtgtgagaatttctgcaactgggaccaaaatgtctttcatagatccttatcagcacattatagtggaacaccagtactcccataagtttactgttgtggttctacgtgccaccaaagtaaccaaggggacctttggcgatatgctggacactccagatccttatgtggaacttttcatctctacaacccctgacagcaggaagcgaacgagacacttcaataatgatataaaccccgtgtggaatgagacctttgagttcattttggatcctaatcaggaaaatgttttggagatcacactgatggatgccaactacgtcatggatgaaaccctaggcacagctacattccctgtatcttcaatgaaagtgggagagaagaaagaagtcccttttattttcaaccaagtcactgaaatgattctggaaatgtctctggaagtttgttcatgcccagacctacggttcagcatggcactgtgtgatcaggagaaaactttcagacagcagaggaaagagaacataaaagagaacatgaagaaacttttgggtccaaaaaagagtgaggggctttattccacacgtgatgtgccggtggtggccattttgggttcaggtgggggtttccgggccatggtgggattctctggtgtgatgaaggcactgtatgagtcggggattttggactgtgctacatacattgctggtctttctggatccacatggtacatgtcaaccttgtactctcaccccgattttccagagaaaggtcccgaggagattaatgaagagctaatgaaaaatgtcagccacaaccctctcttacttcttacaccacagaaagttaaaagatacgttgagtctttatggaagaagaaaagttctggccagcctgtcacctttactgacatctttgggatgctaataggagaaacactaattcaaaataggatgagcatgaccctgagtagtttgaaggaaaaggtcaatgccgcccggtgtcctttgcctctcttcacgtgtctccacgtcaaacctgatgtgtcagagctgatgtttgccgattgggtggaatttagtccatatgagattggcatggcaaaatatggtacctttatggctcctgacctatttggaagcaagttttttatgggaacagttgtaaaaaaatatgaagaaaaccccttgcatttcttgatgggtgtctggggcagtgccttttctatactgttcaacagagttttgggagtttctggctcacagaataaaggctctacaatggaagaggaattagaaaatattacagcaaagcacatcgtgagtaatgacagctccgacagtgatgatgaggctcaaggacccaaaggcaccgagaatgaagaagctgaaaaagagtaccaaagcgacaaccaagcaagttgggtccatcggatgctaatggccttggtgagcgactcggctttattcaatacccgagaaggacgtgccggaaaggtgcataacttcatgctgggcttgaatctcaacacatcatatccactgtctcccctgagagacttcagctctcaggattccttcgatgacgagctcgacgcagcggtagcagatccagatgaatttgaacgaatatatgaaccactggatgtcaaaagtaagaagattcatgtggtagatagtgggctcacatttaacctgccatatcccttgattcttcgacctcagagaggtgtggatcttatcatctcctttgacttttctgcaaggccgagtgacaccagtccccctttcaaggaacttctgcttgcagagaagtgggcgaaaatgaacaagcttccctttccaaagatcgatccttatgtgtttgatcgggaaggattaaaggaatgctatgtttttaaacctaagaatcctgatgtggagaaggattgcccaaccattatccactttgttctggccaacatcaacttcagaaagtacaaggccccaggtgttctaagggaaaccaaagaagagaaagaaattgctgactttgacatttttgatgaccccgaatcgccattttcaaccttcaactttcagtatcccaatcaagcattcaaaaggcttcacgatttgatgtacttcaacacactgaacaacattgatgtgataaaggatgccattgttgagagcattgaatacagaagacagaacccatctcgttgctctgtttccctcagtaatgttgaagcaagaaaattcttcaataaggagtttctaagtaaacccactgtgtaatttctgtgctgggatgatcaagccatttgaattccatgacaatttgagttcagaagacattagaggtcatcttactatgcagaagagactggctgctactcaaagttgtggagatttagccatgtgttaggtgaaaatgatgttgattatgtaatacttagcaacagtttctgacagtatgaattttttgacattagcatagagctatatactgtattttaaacattcctcacattttttacctgtactttttatataaatatgacatgtcttttcttttgaaaatatttaatagtttaactcagtaaaggagacttcccattgtgtgtgaatgttattctgaactagatttgttcatgccatgttacaacactatttttatttaaatgtttatatttacacatacgaaataaatactttgctgtacaaatt

SEQ ID NO: 4 - protein, 748 aa, Mus musculus
MSFIDPYQHIIVEHQYSHKFTVVVLRATKVTKGTFGDMLDTPDPYVELFISTTPDSRKRTRHFNNDINPVWNETFEFILDPNQENVLEITLMDANYVMDETLGTATFPVSSMKVGEKKEVPFIFNQVTEMILEMSLEVCSCPDLRFSMALCDQEKTFRQQRKENIKENMKKLLGPKKSEGLYSTRDVPVVAILGSGGGFRAMVGFSGVMKALYESGILDCATYIAGLSGSTWYMSTLYSHPDFPEKGPEEINEELMKNVSHNPLLLLTPQKVKRYVESLWKKKSSGQPVTFTDIFGMLIGETLIQNRMSMTLSSLKEKVNAARCPLPLFTCLHVKPDVSELMFADWVEFSPYEIGMAKYGTFMAPDLFGSKFFMGTVVKKYEENPLHFLMGVWGSAFSILFNRVLGVSGSQNKGSTMEEELENITAKHIVSNDSSDSDDEAQGPKGTENEEAEKEYQSDNQASWVHRMLMALVSDSALFNTREGRAGKVHNFMLGLNLNTSYPLSPLRDFSSQDSFDDELDAAVADPDEFERIYEPLDVKSKKIHVVDSGLTFNLPYPLILRPQRGVDLIISFDFSARPSDTSPPFKELLLAEKWAKMNKLPFPKIDPYVFDREGLKECYVFKPKNPDVEKDCPTIIHFVLANINFRKYKAPGVLRETKEEKEIADFDIFDDPESPFSTFNFQYPNQAFKRLHDLMYFNTLNNIDVIKDAIVESIEYRRQNPSRCSVSLSNVEARKFFNKEFLSKPTV

SEQ ID NO: 5 - protein, 21 aa, Mus musculus
MEVGWYRSPFSRVVHLYRNGK

(19) United States
(12) Patent Application Publication
(10) Pub. No.: US 2006/0058225 A1
(43) Pub. Date: Mar. 16, 2006

(54) Used of inhibitors of phospholipase a2 for the treatment, prevention or diagnosis of neural inflammatory or demyelinating disease

(75) Inventors: Samuel David, Dorval (CA); Athena Kalyvas, Pierrefonds (CA)

Correspondence Address: FETHERSTONHAUGH - SMART & BIGGAR, 1000 DE LA GAUCHETIERE WEST, SUITE 3300, MONTREAL, QC, H3B 4W5, CA

(73) Assignee: McGill University

(21) Appl. No.: 10/516,241

(22) PCT Filed: May 30, 2003
(86) PCT No.: PCT/CA03/00813

§ 371 Date: Jun. 22, 2005

Related U.S. Application Data
(63) Continuation-in-part of application No. 10/157,898, filed on May 31, 2002, now abandoned.

(30) Foreign Application Priority Data
May 31, 2002 (CA) 2,388,659

(51) Int. Cl. A61K 38/16; A61K 31/739; A61K 48/00; A61K 31/66; A61K 31/12

(52) U.S. Cl. 514/8; 514/125; 514/678; 514/44; 514/54; 514/602

(57) ABSTRACT

The present invention provides methods of preventing and treating neural inflammatory or demyelinating disease, such as multiple sclerosis, via an inhibition of the activity or expression of phospholipase A₂. The invention further relates to methods of identifying phospholipase A₂ inhibitors and their use thereof for the prevention and/or treatment of neural inflammatory or demyelinating disease. An observed increase in the amount of phospholipase A₂ in neural lesions in the EAE animal model system indicates that elevated phospholipase A₂ activity or levels correlate with neural inflammatory or demyelinating disease. Therefore, in a further aspect the invention provides methods for the diagnosis and prognostication of neural inflammatory or demyelinating disease, such as multiple sclerosis.

### FIELD OF THE INVENTION

The invention relates to phospholipase A₂ expression and activity and uses thereof for diagnosis, prognostication, prevention and treatment of neural inflammatory and/or demyelinating disease.

### BACKGROUND OF THE INVENTION

Etiology and pathogenesis of MS and EAE

Multiple sclerosis is an inflammatory demyelinating disease, which typically strikes young adults, and is characterized by demyelinating episodes ranging from relapsing-remitting to chronic progressive in nature. The lesions are multi-focal and confined to the central nervous system (CNS) which includes the brain, spinal cord and optic nerve. Despite extensive studies, the etiology of the disease still remains obscure and its pathogenesis is not fully understood. The consensus is that unknown environmental agent(s) initiate the disease in genetically susceptible individuals. Several genes are thought to be involved in conferring susceptibility to MS. These include HLA class II (likely the DR2, DQ6 locus) (Tienari, 1994) and the T-cell receptor (TCR) genes (Tienari, 1994). However, a definite set of genetic markers for MS remains unknown. Nevertheless, genetic factors are thought to be important contributors to the onset of the disease because MS shows familial clustering and racial differences in risk (Oger and Lai, 1994; Sadovnick et al., 1996; Ebers, 1996).

A number of environmental factors have also been suspected in MS, such as viral and bacterial infections. Elevated antibody titers against a number of viruses have been reported in the cerebrospinal fluid (CSF) and serum of MS patients (Allen and Brankin, 1993). However, viruses have not been detected in the CNS parenchyma in MS.

MS is studied using the established, generally accepted animal model system of experimental allergic encephalomyelitis (EAE), in for example rodents such as rats and mice (Ruuls et al, 1996; Ewing and Bernard, 1998; van der Meide et al, 1998, Smith et al, 2000. As with MS, EAE is also more easily induced in certain strains of mice and rats.

Target Autoantigens and Cytokines in MS and EAE

An important clue to the pathogenesis of MS is the detection of myelin basic protein (MBP)-reactive T-cells in MS in plaques. Injection of MBP peptides into experimental animals can induce EAE (Richert et al., 1989; Martin et al., 1990). Different regions of MBP are encephalogenic in different animal species, e.g., residues 87-106 in Lewis rats and SJL mice, and 1-9 in PL/J and B10.PL mice. Strong evidence for MBP and additional environmental agents in the pathogenesis of MS comes from studies showing that transgenic mice expressing TCR specific for MBP spontaneously develop EAE but only when exposed to a non-sterile environment (Goverman et al., 1993). Thus, exposure to some infectious agent(s) triggers the breakdown of myelin resulting in the availability of MBP and other myelin components for presentation to the TCR via antigen-presenting cells. T-cells that secrete interferon gamma (IFNγ) with reactivities to MBP, PLP (proteolipid protein), MOG (myelin-oligodendrocyte glycoprotein), and MAG (myelin-associated glycoprotein) have been detected in the CSF of MS patients (Olsson et al., 1990; Sun et al., 1991; Zhang et al., 1993). Anti-PLP antibodies have been detected in about 3% of MS patients (Warren et al., 1994), and PLP has been shown to be encephalitogenic (Tuohy, 1994). Studies also show that MOG may be as effective as MBP or PLP in the pathogenesis of MS and EAE (Adelmann et al., 1995; Johns et al., 1995). Thus, a number of CNS myelin components may serve as target autoantigens.

It is thought that autoantigen specific T-cells sensitized in the periphery migrate into the CNS where they initiate the inflammatory changes leading to CNS tissue damage and functional impairment (Bansil et al., 1995). EAE can be induced by injecting mice with MOG or MBP or by the passive transfer of T-cells from affected animals (Moktarian et al., 1984; Zamvil et al., 1985). The findings to date may be taken to indicate that an initial breakdown of myelin by some yet unknown cause, results in the release of myelin components which are then presented by antigen presenting cells to T-cells with receptor specificity for MBP or other myelin antigens. These interactions result in a variety of immune cell responses leading to antibody production and cytotoxicity.

Proinflammatory cytokines such as IFN-γ, TNF-α and β, IL-12 and IL-1β also increase in the CNS of rats with EAE (Issazadeh et al., 1996) and in the brain in MS (Hofman et al., 1989). IL-2 and IFN-γ mRNA levels were shown to be increased in CSF cells from SJL/J mice during MBP-induced EAE (Renno et al., 1994). IFN-γ increases the severity of the rate of relapse in patients with MS (Panitch et al., 1987). TNF-α and β are present in acute and chronic lesions (Hofman et al., 1989). Furthermore, transgenic mice over expressing TNF-α develop a chronic inflammatory demyelination (Probert et al., 1995; Taupin et al., 1997), although other studies on TNF null mice showed similar results (Liu et al., 1998). There are strong similarities in the pathogenesis of MS and EAE (Ewing and Bernard, 1998). As such, EAE is a generally accepted animal model system for MS, and studies on EAE animals have contributed significantly to the understanding of the involvement of the cellular and humoral immune responses in MS (Ewing and Bernard, 1998).

Pathology of MS and EAE Lesions

The CNS lesions in MS and EAE are characterized by widespread focal lesions particularly in perivascular, periventricular and subpial white matter. The pathology varies in acute and chronic lesions. Demyelination is a characteristic feature of acute MS lesions. However, the loss of oligodendrocytes in acute lesions is variable (Bruck et al., 1994; Ozawa et al., 1994). Loss of myelin and oligodendrocytes is more extensive in chronic stages (Prineas et al., 1993; Bruck et al., 1994; Ozawa et al., 1994). The focal lesions also contain inflammatory infiltrates, which consist of T cells and macrophages. In chronic lesions, there is a significant increase in the number of antibody producing plasma cells (Ozawa et al., 1994). CD4⁺ T-cells are found at the edge of the lesions, while macrophages are numerous in and around MS lesions (Traugott et al., 1983; Bo et al., 1994). Activated T-cells are also present in the lesion (Hofman, et al., 1986). Many of these changes in inflammatory cell influx is also seen in EAE lesions in the CNS (Norton et al., 1990; Ewing and Bernard, 1998). However, the inflammatory changes in the CNS rather than demyelination are more prominent in EAE.

Another major pathological feature of MS and EAE is axonal damage (Bjartmar and Trapp, 2001; Trapp et al., 1998; Wujek et al., 2002). The exact mechanism for demyelination and axonal injury in EAE lesions is not known. One possible mechanism is through the destruction of oligodendrocytes. Once the axons are demyelinated they can become damaged through the actions of certain cytokines, proteolytic enzymes and nitric oxide (NO) (Bjartmar and Trapp, 2001; Smith et al., 1999).

Current Approaches in EAE and MS Therapy

Several experimental approaches have been tested in an effort to ameliorate EAE symptoms. Most of these involve immune modulation. These include treatments to block various chemokines or cytokines. Studies performed in blocking chemokines involve the development of anti-MIP-1α, anti-MCP-1 and anti-IP-10 antibodies for treatment of EAE. When the treatment was given before the occurrence of clinical symptoms, anti-MIP-1α antibodies reduced disease incidence by 80% and decreased-disease severity from a clinical score of 2.6 in untreated mice to a score of about a 0.5 in treated mice. When this treatment was given after symptoms began, the severity decreased to a score of 1.25. The anti-MCP-1 treatment only had a minimal effect (Karpus et al, 1995). Treatment with anti-IP-10 tested in only a small experimental group decreased incidence by about 65% and reduced the clinical score to a 0.8 compared to a 3.9 in untreated animals (Fife et al, 2001).

Treatments used to block cytokines have also been tested. These involve blocking lymphotoxin, TNF and IFN-γ. In mice given anti-LT/TNFα antibodies before symptoms appeared reduced disease severity from a score of 3.9 in controls to a 0.2 in treated animals (Ruddle et al, 1990). Similar studies using a TNF binding protein completely prevented EAE in animals treated before symptoms were seen. When this treatment was given after symptoms occurred, the treated animals followed a course from a grade 2 to 0, while control animals went from a grade 2 to 3 to 1 (Selmaj, et al, 1998). Treatments performed using anti-IFN-γ antibodies actually worsened disease severity (Leonard et al, 1996).

Other immunomodulatory treatments evaluated were those done to prevent the actions of macrophages and T cells. Animals treated with liposomes (Cl₂MDP) to eliminate macrophages showed a 40% reduction of EAE incidence (Tran, et al, 1998; Bauer et al, 1995, Huitinga et al, 1990). Disease severity was also reduced, from a mean clinical score of 3.4 in controls to a 0.8 in treated animals, when treatment was given before symptoms occurred (Huitinga et al, 1990). Another method to prevent the actions of lymphocytes is to prevent their entry into the CNS by blocking adhesion molecules at the blood-brain barrier. Studies such as these have been performed using antibodies to ICAM-1, LFA-1 and the α4 integrin. Animals treated with anti-ICAM1 or anti-LFA 1 did not show a significant effect in disease reduction. When they were combined, however, their effect reduced a score of 2.5 in control animals to below 0.5 in the treated group (Kawai et al, 1996). Treatments using anti-α4 integrin antibodies reduced clinical incidence by 75% (Yednock et al, 1992) and reduced disease severity from a 1.5 to a 0.5 (Kent et al, 1995). Other experiments in attempts to block proper T-cell activation and function were also performed. The use of the copper chelator, cuprizone, was used to block IL-2 synthesis and therefore T-cell activation. Treated mice showed decreased disease severity from a score of 4.3 in controls to a 3.3 in mice treated one week before EAE induction. Piperonyl butoxide, an insecticide that is known to deplete T cells delivered before symptoms occurred reduced disease score from a 4.2 in controls to a 2.2. Animals treated after symptoms occurred showed reduced severity to 3.7 (Emerson, et al, 2001).

Oral tolerance has also been evaluated as a treatment for EAE. By feeding animals with myelin antigens, a Th2 response is elicited while Th1 inflammatory responses are reduced. An 80% reduction of EAE incidence was reported in animals fed MBP prior to disease induction. In addition, disease severity was reduced from a maximum score of 4 in control to a 1.4 in treated rats (Popovich et al, 1997). In mice, disease severity was reduced from a 1.6 in control to a 0.6 in treated animals (Meyer et al, 1996). Other methods of switching the Th1 inflammatory cell response to a Th2 cell type response have also been extensively studied. One such treatment is with estrogen. It is known that in pregnant women there is a switch from a Th1 to a Th2 response because of increased levels of this hormone. Mice treated with estrogen showed about a 30% reduction of EAE incidence, a delay of disease onset of about 10 days, and a reduction in disease severity from a 4.5 in untreated animals to a 1.5 (Ito et al, 2001). Mesopram, a type IV phosphodiesterase-specific inhibitor, has also been shown to produce a Th1 to Th2 switch. EAE was prevented in rats treated before the onset of symptoms. Mice treated starting at the first signs of clinical symptoms showed a reduction of a mean clinical score of 4.7 in control animals to a 2.7 in treated animals (Dinter, et al, 2000).

Retinoids, which are ligands of the steroid receptor superfamily, are also thought to favor Th2 cytokine production. They are also thought to increase TGFβ secretion, which is immunosuppressive. When retinamide was given prior to EAE induction, control animals reached a mean clinical score of 3 during relapses, while treated animals went up only to a grade 2 but came down to a 0.75 with no sign of relapse. When retinamide was given after disease symptoms appeared, control animals went from a grade 3.5 to a grade 3 with relapse while treated animals went from a grade 4 to a grade 2 with no relapse (Racke, et al, 1995). Interferon is another molecule thought to serve an immunomodulatory function. Treatment of mice with IFNβ decreases the amount of relapse/mouse from 2.17 in controls to 1.17 in treated animals. Disease severity was also reduced. Control animals progressed from a 3.5 to a 3.8 while treated animals showed a mean clinical score of 3.0 reducing down to a 2.5 (Yu, et al, 1996).

Many signaling pathways are involved in the complex immune reactions seen in EAE and MS. Various kinases are needed to turn-on many of these pathways. Tyrosine kinases mediate the activation of various molecules such as TNFα, prostaglandins (PGE2), and nitric oxide. Tyrosine kinase-blockers have therefore also been evaluated as a possible treatment strategy. These studies have shown about a 60% reduction in incidence of EAE. Also, disease severity was decreased in animals treated before symptoms were seen from a mean clinical score of 3 in controls to a 0.5 in those which received the inhibitor. Mice treated after symptoms occurred reduced severity from a 3 to a 1.5 (Brenner, et al, 1998).

Recent efforts have also focused on decreasing axonal damage in EAE. One way to do this is to reduce the amount of oxidative stress. An inhibitor of inducible nitric oxide synthase (iNOS) given to mice before EAE symptoms appeared decreased symptoms from a 1.3 mean score in controls to a 0.5 in treated animals (Brenner et al, 1997). Metallothinine (MT) is thought to protect cells from reactive oxygen species. Rats treated with MT-II starting at the day of onset of symptoms reduced the score from a 4.5 in controls to a 2 in treated animals (Penkowa and Hidalgo, 2000).

Another way to reduce axonal damage is by blocking glutamate production, which can damage oligodendrocytes and myelin. Experiments using the AMPA/kainate glutamate receptor antagonist NBQX reduced severity from a score of 3 in controls to a 1.5 in treated animals (Smith et al, 2000; Pitt et al, 2000), while MPQX resulted in a greater reduction from a score of 3 to a 0.8. Treating mice during recovery reduced the occurrence of a relapse (Smith et al, 2000).

Of these efforts to develop new treatments for MS, only a few have been approved and are in use (Steinman, 1999; Polman and Uitdehaag, 2000; Rolack, 2001). MS therapies currently being used consist of immunomodulatory drugs such as corticosteroids, Interferon beta, and Glatiramer acetate. Corticosteroids have anti-inflammatory and immunosuppressive effects, which also transiently restores the blood-brain barrier (Noseworthy et al, 2000). They shorten the duration of the relapse and accelerate recovery. Since they are only effective as a short-term treatment, they are most commonly used to treat an acute relapse (Anderson and Goodkin, 1998; Bansil et al, 1995). Further, the responsiveness to corticosteroids declines over time, and extended use may lead to adrenal suppression, cardiovascular collapse and arrhythmias. (C. F. Lacy, L. L. Armsrtong, M. P. Goldman, L. L. Lance. Drug information hand book 8^{th} Edition, 2001, 549-551).

Interferonβ has been used as a therapy for patients with active Relapsing/Remitting Multiple Sclerosis (RRMS) since the 1980's. It is recently being used for secondary progressive patients as well. The exact mode of action of this drug is not yet known. It is thought to play an immunomodulatory role by suppressing T cell mediated inflammation (Stinissen et al, 1997). Recombinant IFNβ is available in 3 drugs: IFNβ-1b (Betaseron) and two IFNβ-1a preparations (Avonex and Rebif) (Polman and Uitedehaag, 2000). These drugs reduce rate of clinical relapse. However, neutralizing antibodies develop against these drugs rendering them ineffective with time. Also, flu-like symptoms are a prominent side effect early on in the treatment.

Glatiramer acetate (copaxone) is a synthetic co-polymer of tyrosine, glutamate, alanine and lysine, thought to mimic MBP and thus, block T cell recognition of MBP (Steinman, et al, 1994). This drug is therefore beneficial in RRMS but not progressive MS. This drug also decreases the rate of relapse and appears to be better tolerated by patients than interferon therapy. Further, treatment with this drug may cause cardiovascular problems such as chest pain, flushing and tachycardia, and respiratory problems such as dyspnea. (C. F. Lacy, L. L. Armsrtong, M. P. Goldman, L. L. Lance. Drug information hand book 8^{th} Edition, 2001, 777-779)

Recently, another drug that has been approved for the use in RRMS and secondary progressive MS is mitroxantrone. This drug is used to arrest the cell cycle and prevent cellular division. It is primarily used in leukemias (Rolak, 2001). In MS it reduces relapse rate and increases the length between exacerbations. This drug however has long-term side effects causing cardiac toxicity. Another treatment that has limits to its usefulness is intravenous immunoglobulin. It acts to alter the immune system in a beneficial way and it has shown to cut relapses in half (Rolak et al, 2001). However, the treatments are very expensive.

As discussed above, there are a few moderately effective treatments for RRMS and secondary progressive MS that have shown to reduce both the frequency of the disease and severity of exacerbations. However, problems still exist in treating MS, and there are still no proven treatments, for example, for primary progressive MS. There is therefore a continued need for improved materials and methods for the treatment of neurodegenerative diseases such as MS.

The area of MS diagnosis is significantly less developed, as no measurable biochemical/genetic markers of the disease state exist. As a result, MS diagnosis relies on examining the pathology of the affected tissue by Magnetic Resonance Imaging (MRI) methods. MRI is very costly, and as such its availability is severely limited, typically leading to long waiting lists for testing. Increased cost also limits availability of MRI equipment and expertise to larger communities, thus necessitating travel for those patients residing elsewhere. Further, to justify performing such a costly test, patients are chosen which appear to already exhibit relatively severe symptoms associated with MS, and as such this type of diagnosis is performed significantly later than disease onset, and thus does not provide the opportunity for earlier detection and treatment. There therefore further exists a continued need for improved methods and materials for the diagnosis and prognostication of neurodegenerative diseases such as MS.

### SUMMARY OF THE INVENTION

The invention relates to phospholipase A₂-based therapeutic, prophylactic, diagnostic and prognostic methods; related screening methods, as well as related uses and commercial packages.

Accordingly, in a first aspect, the invention provides a method of preventing or treating a neural inflammatory or demyelinating disease in an animal, said method comprising inhibiting the activity of a phospholipase A₂ in the animal. In an embodiment, the method comprises administering to the animal an effective amount of a phospholipase A₂ inhibitor.

In an embodiment, the method comprises inhibiting the expression of a phospholipase A₂.

The invention further provides a use of a phospholipase A₂ inhibitor, or a composition comprising a phospholipase A₂ inhibitor in admixture with a pharmaceutically acceptable carrier, for preventing or treating a neural inflammatory or demyelinating disease in an animal.

The invention further provides a use of a phospholipase A₂ inhibitor for preparation of a medicament for preventing or treating a neural inflammatory or demyelinating disease in an animal.

The invention further provides a commercial package comprising a phospholipase A₂ inhibitor together with instructions for preventing or treating a neural inflammatory or demyelinating disease in an animal.

The invention further provides a method of assessing a neural inflammatory or demyelinating disease in an animal, said method comprising: (a) determining a test level of phospholipase A₂ protein or phospholipase A₂ encoding mRNA or phospholipase A₂ enzyme activity in tissue or body fluid of the animal; and (b) comparing said test level of phospholipase A₂ protein or phospholipase A₂ encoding mRNA or phospholipase A₂ activity to an established standard; or to a corresponding level of phospholipase A₂ protein or phospholipase A₂ encoding mRNA or phospholipase A₂ enzyme activity in tissue or body fluid of a control animal; or to a corresponding level of phospholipase A₂ protein or phospholipase A₂ encoding mRNA or phospholipase A₂ enzymatic activity in tissue or body fluid obtained from said animal at an earlier time; wherein an increase in said test level is indicative of the neural inflammatory or demyelinating disease.

The invention further provides a commercial package comprising means for determining the level of phospholipase A₂ protein or phospholipase A₂ encoding mRNA or phospholipase A₂ enzyme activity in a tissue or body fluid of an animal together with instructions for assessing a neural inflammatory or demyelinating disease. In an embodiment, the instructions comprise: (a) determining, using said means, a test level of phospholipase A₂ protein or phospholipase A₂ encoding mRNA or phospholipase A₂ enzyme activity in tissue or body fluid of the animal; and (b) comparing said test level of phospholipase A₂ protein or phospholipase A₂ encoding mRNA or phospholipase A₂ activity to an established standard; or to a corresponding level of phospholipase A₂ protein or phospholipase A₂ encoding mRNA or phospholipase A₂ enzyme activity in tissue or body fluid of a control animal; or to a corresponding level of phospholipase A₂ protein or phospholipase A₂ encoding mRNA or phospholipase A₂ enzymatic activity in tissue or body fluid obtained from said animal at an earlier time; wherein an increase in said test level is indicative of the neural inflammatory or demyelinating disease.

In an embodiment, the tissue or body fluid is selected from the group consisting of blood, plasma, cerebrospinal fluid, neural cells, endothelia, and immune cells (e.g. macrophages, leukocytes and lymphocytes).

In an embodiment, the animal is a mammal, in a further embodiment, a human.

In an embodiment, the neural inflammatory or demyelinating disease is selected from the group consisting of Multiple Sclerosis, Alzheimer's disease, amyotrophic lateral sclerosis and stroke.

In an embodiment, the phospholipase A₂ is a cytosolic phospholipase A₂.

In an embodiment, the above-noted inhibitor is selected from the group consisting of arachidonic acid analogues, benzenesulfonamide derivatives, bromoenol lactone, p-bromophenyl bromide, bromophenacyl bromide, trifluoromethylketones, sialoglycolipids and proteoglycans. In a further embodiment, the inhibitor is selected from the group consisting of arachidonyl trifluoromethyl ketone, methyl arachidonyl fluorophosphonate, palmitoyl trifluoromethyl ketone.

In a further embodiment, the phospholipase A₂ inhibitor is selected from the group consisting of an antisense molecule and an siRNA or siRNA-like molecule.

In an embodiment, the antisense molecule is a nucleic acid that is substantially complementary to a portion of an mRNA encoding a phospholipase A₂. In an embodiment, the antisense molecule is complementary to a portion of a nucleic acid sequence substantially identical to a sequence selected from the group consisting of SEQ ID NO:1 and SEQ ID NO:3. In an embodiment, the portion of an mRNA comprises at least 5 contiguous bases.

In an embodiment, the siRNA or siRNA-like molecule is substantially identical to a portion of an mRNA encoding a phospholipase A₂. In an embodiment, the siRNA or siRNA-like molecule is substantially identical to a portion of an mRNA corresponding to a DNA sequence selected from the group consisting of SEQ ID NO:1 and SEQ ID NO:3. In an embodiment, the siRNA or siRNA-like molecule comprises less than about 30 nucleotides, in a further embodiment, about 21 to about 23 nucleotides.

The invention further provides a method of identifying a compound for prevention and/or treatment of neural inflammatory and/or demyelinating disease, said method comprising: (a) providing a test compound; and (b) determining whether activity or expression of a phospholipase A₂ is decreased in the presence of said test compound; wherein a decrease in said activity is indicative that said test compound may be used for treating a neural inflammatory and/or demyelinating disease. In an embodiment, the method further comprises the step of assaying the compounds for activity in the prevention or treatment of a neural inflammatory or demyelinating disease.

The invention further provides a method of identifying or characterizing a compound for prevention or treatment of a neural inflammatory or demyelinating disease, said method comprising: (a) contacting a test compound with a cell comprising a first nucleic acid comprising a transcriptionally regulatory element normally associated with a PLA₂ gene, operably linked to a second nucleic acid comprising a reporter gene capable of encoding a reporter protein; and (b) determining whether reporter gene expression or reporter protein activity is decreased in the presence of said test compound; said decrease in reporter gene expression or reporter protein activity being an indication that said test compound may be used for prevention or treatment of neural inflammatory or demyelinating disease.

In an embodiment, the phospholipase A₂ is a mammalian phospholipase A₂, in a further embodiment, a human phospholipase A₂. In an embodiment, the phospholipase A₂ is a cytosolic phospholipase A₂.

In an embodiment, the neural inflammatory or demyelinating disease is selected from the group consisting of Multiple Sclerosis, Alzheimer's disease, amyotrophic lateral sclerosis and stroke.

### BRIEF DESCRIPTION OF THE DRAWINGS

FIG. 1: Schematic illustration of PLA₂ enzyme activity.

FIG. 2: Endothelial cells in EAE lesions express cPLA₂. Spinal cord tissue of mice with EAE at clinical grades 1-4. Arrows indicate cPLA₂⁺ elongated cells in EAE lesions. cPLA₂ positive cells are seen in grades 1-3. No cPLA₂ labeling of endothelial cells is seen in grade 4 micrograph. Slides were counterstained with methyl green which gives a grey staining in the black and white pictures.

FIG. 3: Immune cells in EAE infiltrates also express cPLA₂. cPLA₂⁺ immune cells in the infiltrates were seen at all clinical grades. Arrows point to positive immune cells. Slides counterstained with methyl green.

FIG. 4: Changes in the number of cPLA₂⁺ endothelial cells in EAE lesions. High numbers of endothelial cells, between 45%-85% express cPLA₂ during the earlier stages of the disease, i.e., at grades 1 to 3. The numbers peaked at 85% in grade 3 and reduced to less than 20% at grades 4 and 5.

FIG. 5: Changes in the number of cPLA₂⁺ immune cells in EAE lesions. Between 25% to 50% of the immune cells in the CNS infiltrates were cPLA₂+at all clinical grades.

FIG. 6: Histogram showing total numbers of immune cells in EAE lesions. Total number of immune cells infiltrating the CNS at different clinical grades. The number of cells in EAE lesions increases at grades 4 and 5.

FIG. 7: Total number of cPLA₂⁺ immune cells in EAE lesions at different clinical grades. The total number of cPLA₂ positive immune cells increases at later grades of 4 and 5.

FIG. 8: Cell types expressing cPLA₂ in EAE lesions The cell types expressing cPLA₂ in the spinal cords were assessed using double immunofluorescence. GFAP⁺ astrocytes (row 1), CD34+endothelial cells (row 2), Mac-1⁺ macrophages (row 3) and CD4⁺ T cells (row 4) were cPLA₂⁺ at and near the EAE lesions. Double labelling of these cells is shown in the column labelled “merge”.

FIG. 9: Incidence of EAE. In the control group, 100% of the mice showed clinical signs of EAE induced paralysis. In contrast to the controls (ctl), mice treated with either 2 (low) or 4 (high) mM AACOCF₃ had EAE incidence of 57% and 28%, respectively.

FIG. 10: Clinical course of EAE. Graph showing changes in the clinical course of the disease. EAE was induced in all groups of mice. Mice in the control group (Ctl) that did not receive any treatment reached a peak clinical score of almost 3 at days 12-14 during the first paralytic episode. Compared to the control group, mice treated with 2 and 4 mM AACOCF₃ only reached scores of 1.5 and 0.4, respectively. Furthermore, the control group relapsed into a second paralytic episode between days 26 and 34, while the 4 mM treated group remained unaffected.

FIG. 11: Effect of delayed (i.e. after the peak of the first attack of EAE symptoms) PLA₂ inhibitor treatment of mice. Mice that develop a milder form of the disease, i.e., reach a mean clinical score of 3, while recovering to a grade 2 on day of treatment, show complete recovery and lack of subsequent relapses when treated with 4 mM AACOCF₃ (treat-gr2). In contrast control mice that reach a mean clinical score of 3, while recovering to a grade 2 or less after the first paralytic episode, suffer subsequent paralytic episodes, reaching a mean clinical score of 2.5 (Ctl-gr2).

FIG. 12: Therapeutic effect of delayed (i.e., after the peak of the first attach of EAE symptoms) PLA₂ treatment of mice. Graph showing the clinical course of mice induced with EAE that were treated with 4 injections of 4 mM AACOCF₃ (▪; n=6) between days 14-20 (indicated by arrows) and the untreated control groups (▴; n=9). Treated mice that recovered to a grade 2 or below on day 14, the first day of treatment, show a remarkable recovery and remain almost symptom free until day 60. In contrast, untreated control mice that also recovered to a grade 2 or below on day 14 show a relapsing-remitting clinical course. Note that the second attack which occurs in the controls around day 30 is prevented in the treated mice even though the treatment was stopped at day 20. Differences between these groups are statistically significant between days 28 to 60 (P<0.005).

FIG. 13: Graph showing the clinical course of mice with EAE that remained at a grade 3 or above on day 14, the first day of inhibitor treatment (●; n=7), as well as the untreated control mice (▾; n=8) that also remained at a grade 3 or above. These mice show a chronic clinical course of EAE and did not show signs of clinical improvement with inhibitor treatment.

FIG. 14: Effects on Inflammation. (a) Quantitative analysis of H&E stained sections of the lumbar spinal cord revealed a significant reduction in the number of immune cells entering the spinal cord in both groups (relapsing-remitting and chronic groups shown in FIGS. 12 and 13) of treated mice compared to their untreated controls, *, P<0.02; **P<0.03. Untreated (chronic)=Untreated EAE mice with chronic clinical course that remained at a score of 3 or above on day 14. Treated (chronic) inhibitor treated EAE mice that showed a chronic course that also remained at a score of 3 or above on day 14. Untreated (RR)=untreated EAE mice that show a relapsing-remitting clinical course that had remitted to a sore of 2 or below on day 14. Treated (RR)=inhibitor treated EAE mice that recovered to a grade 2 or below on day 14). (b) There was also a reduction in the number of EAE lesions in the lumbar spinal cord in both treated groups compared to their untreated controls, *, P<0.04; ** P<0.01. (c) A decrease in the area of the EAE inflammatory lesions was also seen in both treated groups versus their controls, *, P<0.01; **, P<0.04.

FIG. 15: Electron micrographs of lumbar spinal cord tissue near the site of EAE lesions in the treated chronic group (a) and their untreated control chronic group (b). Note the large number of degenerating axons in a and b. Scale bar, 30 μm. The micrographs in c, d and e show higher magnifications of degenerating axons indicated by arrows in a. Scale bars, 5 μm.

FIG. 16: Tabular results of studies of AACOCF₃ treatment (NS=not significant).

FIG. 17: Tabular results of studies of delayed AACOCF₃ treatment (NS=not significant).

FIG. 18: Human cPLA₂ DNA sequence (GenBank #: M72393; Sharp et al., 1991).

FIG. 19: Mouse cPLA₂ DNA sequence (GenBank #: M72394; Nalefski et al., 1994)

### DETAILED DESCRIPTION OF THE INVENTION

Although a variety of environmental factors are thought to induce the onset of MS in genetically susceptible individuals, it is proposed herein that these factors likely trigger the activation of a common mechanism that leads to infiltration of immune cells into the CNS, neural tissue damage and myelin breakdown. It is described herein that a likely candidate that could mediate such a common mechanism is the enzyme phospholipase A₂ (PLA₂). One of the metabolic products of PLA₂ is arachidonic acid, which gives rise to eicosanoids such as prostaglandins, thromboxanes and leukotrienes that are potent mediators of inflammatory responses. Another metabolic product of PLA₂ is lysophosphatidylcholine (LPC) which has potent detergent-like properties. Injection of LPC into the CNS and PNS causes myelinolysis (Hall, 1993, Jeffery and Blakemore, 1995; Ousman and David, 2000). LPC is also a chemoattractant for human T cells and monocytes (Ryborg et al., 1994; Prokazova et al., 1998). LPC also induces expression of a number of chemokines and cytokines that are involved in immune cell influx and activation in the CNS (Ousman and David, 2001). Some of these cytokines and chemokines are known to induce the expression of PLA₂. Therefore, LPC produced by PLA₂-mediated hydrolysis of phosphatidylcholine could result in expression of chemokines and cytokines that induce further expression of PLA₂.

Phospholipase A₂

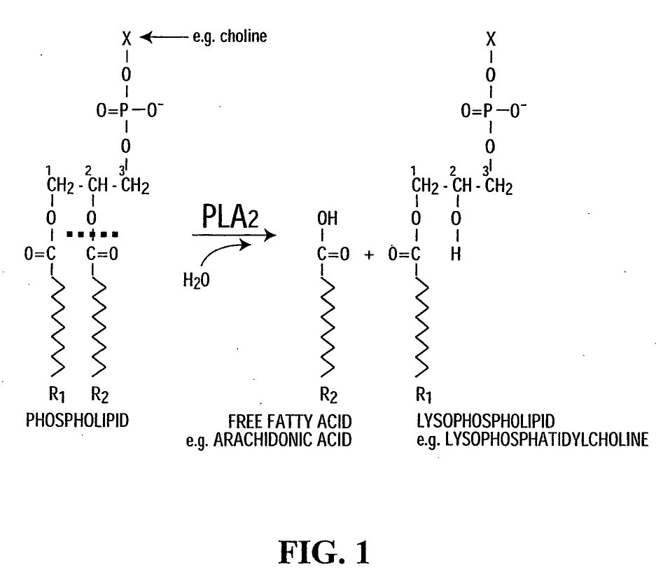

Phospholipase A₂ hydrolyzes the fatty acyl ester bond at the sn-2 position of glycerophospholipids (FIG. 1). The immediate products of a PLA₂-catalyzed reaction are a free fatty acid (e.g., arachidonic acid), and a lysophospholipid (e.g., lysophosphatidylcholine). Phospholipase A₂ has 2 major physiological functions: (1) membrane turnover; (2) potent mediator in the activation of inflammatory processes (Dennis, 1994). Ten different PLA₂ have been identified which fall into two major types: secreted (sPLA₂), and cytosolic (cPLA₂). Various forms of PLA₂ are found in different tissues and cell types or are unique to the venom of reptiles and insects.

Secreted PLA₂: Several forms of sPLA₂ exist all of which have molecular weights of about 14 kDa. Group IB sPLA₂ is the pancreatic form that is secreted in digestive juices. It is not expressed in the CNS. Group IIA sPLA₂ is produced by many other cells of the body including neutrophils, thymus, bone marrow, spleen, astrocytes, Schwann cells, etc., (Kramer et al., 1989; Komada et al., 1989; Ishizake et al., 1989; Wright et al., 1989; Murakami et al., 1990; Nakano and Arita, 1990). Group. IIA sPLA₂ is detected in exudates from sites of inflammation or tissue injury such as ascites fluid suggesting that macrophages are a source (Kramer et al., 1989; Trotter and Smith, 1986; Forst et al., 1986; Chang et al., 1987; Seilhamer et al., 1989). Group IIA sPLA₂ from various sources have been purified. It is expressed widely in the brain (Molloy et al., 1998). Another form of sPLA₂, group V is expressed mainly in the heart, lung and placenta, and in very low levels in the brain, except in the hippocampus where it may play a specific physiological role (Molloy et al., 1998). Group X is found mainly in human leukocytes. Groups IA, IIB and III are found only in certain venoms, and group IX in the marine snail (Dennis, 2000). Pro-inflammatory cytokines such as TNF and IL induce expression of sPLA₂ in cultured astrocytes (Oka and Arita, 1991), chondrocytes (Lyons-Giordano et al., 1989) and vascular smooth muscle cells, (Nakano et al., 1990; Arbibe et al., 1997). In addition, human endothelial cells from the umbilical vein express type II sPLA₂ when treated with TNF (Murakami et al., 1993). sPLA₂ require millimolar concentrations of calcium for their activation. U.S. Pat. No. 6,103,469 (Hawkins et al., Aug. 15, 2000) relates to a sPLA₂. The activity of sPLA₂ can be blocked by p-bromophenacyl bromide (Glaser et al., 1993). Other inhibitors are currently being tested by Eli Lilly in preclinical trials in non-CNS models of inflammation (Ogata et al., 2001).

Cytosolic PLA₂: Three forms of cPLA₂ have been identified in recent years. The calcium-dependent form of cPLA₂ (group IV) is found in a variety of mammalian cells and tissues (Glaser et al., 1993). It has a molecular weight of 85 kDa. Group IV cPLA₂ requires micromolar concentrations of calcium and is widely expressed in the brain (Molloy et al., 1998), as well as in neutrophils and endothelial cells (Arbibe et al., 1997; Fujimori et al., 1992; Lautens et al., 1998). It prefers arachidonic acid at the sn-2 position, which means it is capable of selectively releasing arachidonic acid (Glaser et al., 1993). cPLA₂ is phosphorylated and its activation increased by MAP kinase (Lin et al., 1993). Group IV cPLA₂ has been purified from a variety of cellular sources. U.S. Pat. No. 6,242,206 (Choiu et al., Jun. 5, 2001) relates to a cPLA₂.

cPLA₂ expression is increased in neurons in the hippocampus after transient global ischemia (Owada et al., 1994). In addition, mice deficient in cPLA₂ (group IV) are resistant to cerebral ischemia (Bonventre et al., 1997) and MPTP neurotoxicity (Klivenyl et al., 1998). Like sPLA₂, the expression of cPLA₂ in a variety of cells is increased by pro-inflammatory cytokines such as TNF, IFN-γ, IL-1 and CSF-1 (Hulkower et al., 1992; Goppelt-Struebe and Rehfeldt, 1992; Lin, Lin and DeWitt, 1992; Xu et al., 1994; Wu et al., 1994). It can be inhibited by arachidonic acid analogues such as arachadonyl trifluromethylketone (AACOCF₃) and methyl arachidonyl fluorophosphonate (MAFP) (Dennis, 2000; Glaser et al., 1993). Ross et al. (1995) isolated a 180 kDa calcium-dependent form of cPLA₂ from human brain which could be inhibited by bromophenacyl bromide, as well as, AACOCF₃.

Two calcium-independent forms of cPLA₂ have also been isolated from the bovine brain (Hirashima et al., 1992; Farooqui et al., 1997). The 29 kDa form is inhibited by sialoglycolipids, and various proteoglycans (Yang et al., 1994). Another 80-85 kDa calcium independent form of cPLA₂, which exists in multimeric form of 300 kDa has been identified in macrophages. This form can be inhibited by the arachidonic acid analogue, AACOCF₃. Other calcium-independent forms have been identified in myocardial cells and the brush border of the intestine (Murakami, Nakatani Atsumi et al., 1997) but these are not of relevance to the CNS.

The precise physiological role of the various forms of cPLA₂ in the CNS is not known at present. The studies described herein are particularly interested in the ability of cPLA₂ to induce inflammatory responses via production of arachidonic acid. This activity of various forms of cPLA₂ can be effectively inhibited by the arachidonic acid analogues AACOCF₃ and MAFP (Balsinde et al., 1999). Elevated levels of PLA₂ have been detected in MS tissue in an older study (Woelk and Peiler-Ichikawa, 1974), however, this study was done in vitro utilizing post mortem tissue, and thus provides no indication of conditions in living tissue. Another study found no change in the level of secreted PLA₂ activity in MS samples versus controls, and found a decrease in cytosolic PLA₂ activity in samples from MS subjects (Huterer, Tourtellotte and Wherrett, 1995). Furthermore, the downstream products of arachidonic acid and 5-lipoxygenase, such as leukotriene C₄ are elevated in the CSF of MS patients (Dore-Duffy et al., 1991). Levels of prostaglandins, which are derived from arachidonic acid by the action of cyclooxygenase, also correlate with the severity of MS (Dore-Duffy et al., 1986), and blocking these reduces the severity of EAE in mice (Reder et al., 1994). In addition, TNF and IL-β, which are capable of inducing expression of both forms of PLA₂, are elevated in the CSF of patients with MS (Hauser et al., 1990).

LPC Mediates Chemokine and Cytokine Expression and Immune Cell Responses

LPC, another metabolic product of PLA₂, in addition to being a strong myelinolytic agent is also a chemoattractant for T-cells and monocytes and is mitogenic for macrophages (Ryborg et al., 1994; Prokazova et al., 1998). It has been found that injection of LPC into the adult mouse spinal cord leads to the rapid expression of MCP-1, MIP-1α, GM-CSF and TNF-α as determined by RT-PCR (Ousman and David, 2001). The expression of these chemokines and cytokines mediates the rapid influx of T-cells and monocytes into the spinal cord, and to activation of macrophages (Ousman and David, 2000, 2001). These immune cell changes result in rapid demyelination at the site of LPC injection within the spinal cord in 4 days. Previous work of the applicants' laboratory has shown that LPC also induces increased expression of VCAM-1 and ICAM-1 in blood vessels in the mouse spinal cord, as well as, induces a marked opening of the blood-brain barrier (Ousman and David, 2000). These adhesion molecules are important in mediating the extravasation of leukocytes into the CNS parenchyma in EAE and are also expressed in active MS plaques (Lee and Benveniste, 1999; Sobel, Mitchell and Fondren, 1990; Raine and Cannella, 1992).

Described herein are experiments to assess the expression of cPLA₂ in EAE lesions in the spinal cord in C57BL/6 mice. This mouse strain has a naturally occurring null mutation for the major form of sPLA₂ (Group IIA) (Kennedy et al., 1995). Since EAE can be induced in these mice, it is unlikely that sPLA₂ is the only major inducer of the disease. The expression of cPLA₂ was therefore examined in EAE lesions in the spinal cord of C57BL/6 mice using immunohistochemical techniques. As a result, it is shown herein that cPLA₂ is indeed expressed in higher amounts in such lesions. Experiments were then carried out in which the activity of cPLA₂ was blocked using a chemical inhibitor. These experiments revealed that blocking cPLA₂ prevents the onset and progression of EAE.

Demonstrated herein is an increase in cPLA₂ in and around EAE lesions in C57BL/6 mice that have a natural disruption in the sPLA₂ gene. The increase in cPLA₂ was seen in endothelial cells and astrocytes, whose processes surround blood vessels. A high level of expression in endothelial cells was seen just prior to the highest increase in the influx of inflammatory cells into the spinal cord. cPLA₂ expression was also seen in the T cells and macrophages that accumulate at the site of immune lesions in the spinal cord. Previous studies have shown an increase of downstream products of PLA₂ action such as prostaglandins and leukotrienes in the CSF of MS patients (Gallai et al, 1995; Fretland, 1992), however, a role for PLA₂ has not been described prior to the studies described herein. Animal studies using the EAE model to assess the blocking effects of these downstream products have been shown herein to reduce the severity of EAE. A prostaglandin E1 analogue was shown to delay onset of EAE by a few days and reduce clinical severity from a mean grade of 2.23 in controls to 0.7 in treated rats (Reder et al, 1994). A leukotriene inhibitor, sulfasalazine, also reduced disease incidence in guinea pigs (Prosiegel et al, 1990). A COX-inhibitor, piroxicam, was shown to decrease mean clinical score from a 2.8 in untreated to a 1.5 in treated rats (Weber and Hempel, 1989). In addition, a dual COX/5-lipoxygenase inhibitor was shown to reduce the incidence of EAE (Prosiegel et al, 1989). Provided herein is direct evidence that the use of PLA₂ inhibitors markedly reduces the incidence and severity of EAE. The incidence of EAE using AACOCF₃ was reduced by 72% in treated mice. Also, disease severity was reduced from a mean maximal clinical score of almost 3 in control mice to 0.4 in treated mice.

As described herein, the effects of blocking PLA₂ activity are not only immunosuppressive, but also prevent myelin breakdown. The results described herein demonstrate the effectiveness of this inhibitor in an animal model of MS. Therefore, blocking PLA₂ directly can be used as a new therapeutic tool for MS. By blocking PLA₂ upstream of the arachidonic acid metabolites, both the inflammatory cascade and myelin disruption through LPC will be prevented, leading to a potentially effective treatment for MS.

Further, the results herein demonstrate a role for cPLA₂ in both inflammatory and axonal pathologies seen in EAE. As such, cPLA₂-modulation may be used to treat and/or prevent both inflammation and axonal damage (including demyelination) of a neural inflammatory and/or demyelinating disease such as MS.

Accordingly, in an aspect, the invention provides a method for the prevention and/or, treatment of neural inflammatory and/or demyelinating disease in an animal, the method comprising modulating (in an embodiment, inhibiting) the activity and/or expression of a phospholipase A₂ (PLA₂) in the animal. Such a method may comprise administering to the animal (e.g. an animal in need thereof) an agent capable of modulating PLA₂ activity. In cases involving an inhibition of PLA₂ activity, such an agent is a PLA₂ inhibitor. Such administration may in embodiments occur before, at about the time of, or subsequent to the onset of the disease. An “agent capable of modulating PLA₂ activity” refers to any compound which when introduced into a system comprising a PLA₂ protein, is capable of altering at least one aspect of PLA₂ activity or function. Such an agent may be a ligand of a PLA₂ protein, such as an agonist or antagonist. Such an agent may act directly on a PLA₂ protein or indirectly by modulating a process or activity, which subsequently results in the modulation of PLA₂ activity, or may modulate PLA₂ expression. In certain systems (e.g. in vivo)., such an agent may be a prodrug, which is metabolized to an active form at or prior to its arrival at the site of action.

In another aspect, the invention provides a method for the diagnosis and/or prognostication of neural inflammatory and/or demyelinating disease in an animal, the method comprising determining a level of PLA₂ protein or expression or activity of a PLA₂ in a tissue or body fluid obtained from the animal.

In an embodiment, the therapeutic method of the invention may be used in conjunction with a diagnostic method (such as the diagnostic method of the invention), whereby a subject is first identified to be in need of treatment, and subsequently an agent capable of modulating (e.g. inhibiting) PLA₂ activity may be administered to the subject in need thereof.

In embodiments, the disease is multiple sclerosis and related neural diseases. In further embodiments, the disease is selected from the group consisting of Alzheimer's disease, amyotrophic lateral sclerosis and stroke. In an embodiment, the animal is a mammal, in a further embodiment, a human. In embodiments, the PLA₂ is secreted or cytosolic, calcium dependent or independent. In an embodiment, the PLA₂ is of an average molecular weight of about 14 kDa. In an embodiment, the PLA₂ is of an average molecular weight of about 85 kDa. In an embodiment, the PLA₂ is cytosolic PLA₂ (cPLA₂). In an embodiment, the PLA₂ is a calcium-dependent PLA₂. In embodiments, the PLA₂ is a type IV PLA₂. In embodiments, the method comprises the modulation of both a secreted and a cytosolic PLA₂. In embodiments, the PLA₂ has an activity that generates as a product (a) arachidonic acid (b) lyso-phosphatidylcholine or (c) both (a) and (b).

Chemokines and cytokines, are thought to mediate (play a role in) a variety of disease states. In alternative aspects, the invention relates to methods, uses and commercial packages for immunomodulation (e.g. immunosuppression) and for diagnosis, prognostication, prevention and/or treatment of T-cell mediated diseases, including autoimmune diseases, inflammation, chronic interstitial lung disease, rheumatoid arthritis, inflammatory bowel disease, Crohn's disease, ulcerative colitis, allergy, contact hypersensitivity, psoriasis, systemic lupus erythematosus, osteoarthritis, and diseases mediated by superantigen toxins such as staphylococcal enterotoxin B, and toxic shock syndrome toxin 1.

“Modulation/modulating” as used herein refers to both upregulation (i.e., activation or stimulation (e.g., by agonizing or potentiating)) and downregulation (i.e. inhibition or suppression (e.g., by antagonizing, decreasing or inhibiting)).

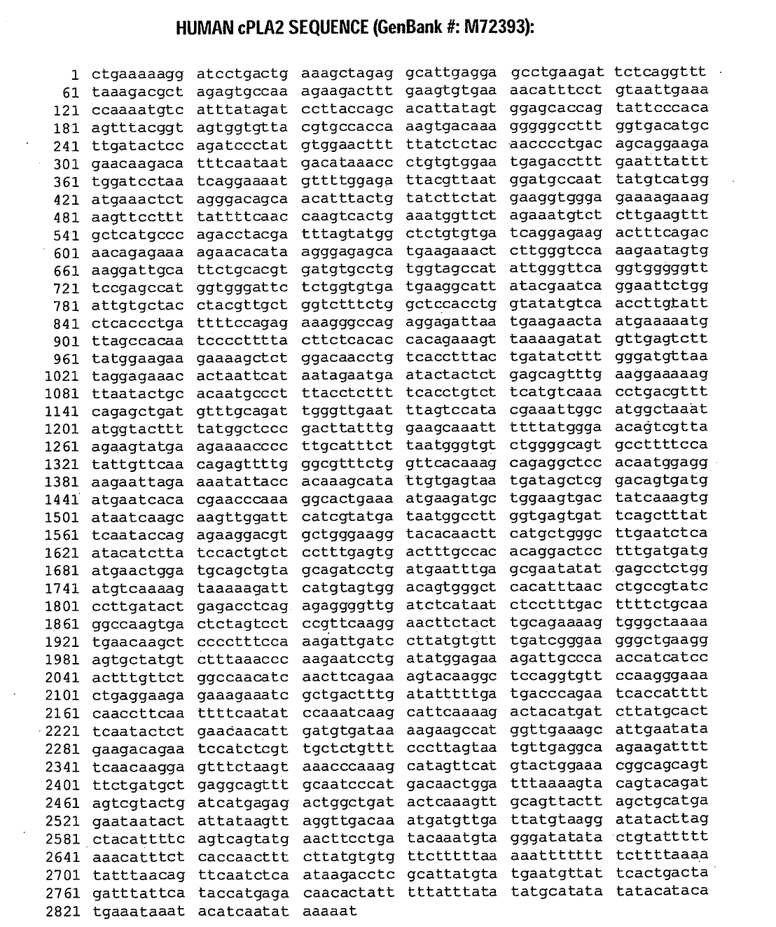

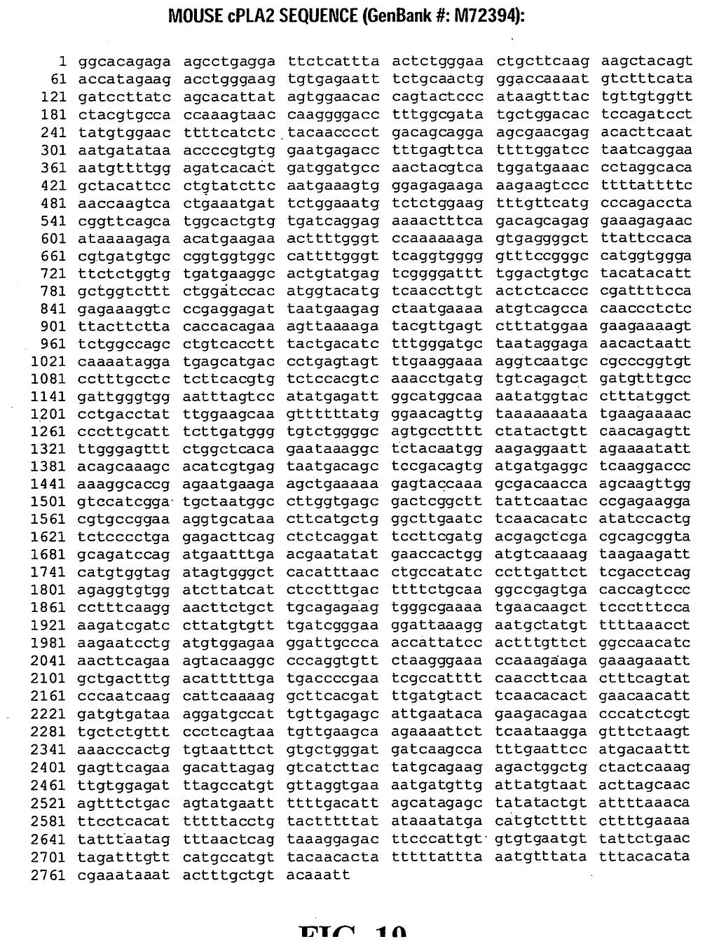

A wide variety of alternative genomic approaches are available to down-regulate the expression of functional PLA₂. For example, in alternative embodiments, transformation of cells with antisense constructs may be used to inhibit expression of PLA₂. Antisense constructs are nucleic acid molecules that may be transcribed to provide an antisense molecule that is substantially complementary to all or a portion of the mRNA encoding PLA₂, so that expression of the antisense construct interferes with the expression of the PLA₂. In an embodiment, the just noted antisense molecule is antisense to a DNA sequence coding PLA₂, in an embodiment, a human PLA₂. Shown in FIG. 18 and SEQ ID NO:1 is a human DNA sequence encoding a cPLA₂ (Sharp et al., 1991), with the putative coding sequence shown in SEQ ID NO:1 and the corresponding cPLA₂ protein sequence shown in SEQ ID NO:2. Shown in FIG. 19 and SEQ ID NO:3 is a mouse DNA sequence encoding a cPLA₂ (Nalefski et al., 1994), with the putative coding sequence shown in SEQ ID NO:3 and the corresponding cPLA₂ protein sequence shown in SEQ ID NO:4. In some embodiments, antisense constructs of the invention may therefore encode five or more contiguous nucleic acid residues substantially complimentary to a contiguous portion a nucleic acid sequence encoding PLA₂, such as an mRNA encoding a PLA₂, or said antisense constructs may encode a sequence of five or more contiguous nucleic acid residues which are antisense to the DNA sequences in SEQ ID NO:1 and/or SEQ ID NO:3.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as. GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 5° C. lower than the thermal melting point for the specific sequence at a defined ionic strength and pH.

In alternative embodiments, the invention provides antisense molecules and ribozymes for exogenous administration to bind to, degrade and/or inhibit the translation of PLA₂ mRNA. Examples of therapeutic antisense oligonucleotide applications, incorporated herein by reference, include: U.S. Pat. No. 5,135,917, issued Aug. 4, 1992; U.S. Pat. No. 5,098,890, issued Mar. 24, 1992; U.S. Pat. No. 5,087,617, issued Feb. 11, 1992; U.S. Pat. No. 5,166,195 issued Nov. 24, 1992; U.S. Pat. No. 5,004,810, issued Apr. 2, 1991; U.S. Pat. No. 5,194,428, issued Mar. 16, 1993; U.S. Pat. No. 4,806,463, issued Feb. 21, 1989; U.S. Pat. No. 5,286,717 issued Feb. 15, 1994; U.S. Pat. No. 5,276,019 and U.S. Pat. No. 5,264,423; BioWorld Today, Apr. 29, 1994, p. 3.

Preferably, in antisense molecules, there is a sufficient degree of complementarity to the PLA₂ mRNA to avoid non-specific binding of the antisense molecule to non-target sequences under conditions in which specific binding is desired, such as under physiological conditions in the case of in vivo assays or therapeutic treatment or, in the case of in vitro assays, under conditions in which the assays are conducted. The target mRNA for antisense binding may include not only the information to encode a protein, but also associated ribonucleotides, which for example form the 5′-untranslated region, the 3′-untranslated region, the 5′ cap region and intron/exon junction ribonucleotides. A method of screening for antisense and ribozyme nucleic acids that may be used to provide such molecules as PLA₂ inhibitors of the invention is disclosed in U.S. Pat. No. 5,932,435 (which is incorporated herein by reference).

Antisense molecules (oligonucleotides) of the invention may include those which contain intersugar backbone linkages such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Oligonucleotides having morpholino backbone structures may also be used (U.S. Pat. No. 5,034,506). In alternative embodiments, antisense oligonucleotides may have a peptide nucleic acid (PNA, sometimes referred to as “protein nucleic acid”) backbone, in which the phosphodiester backbone of the oligonucleotide may be replaced with a polyamide backbone wherein nucleosidic bases are bound directly or indirectly to aza nitrogen atoms or methylene groups in the polyamide backbone (Nielsen et al., 1991, Science 254:1497 and U.S. Pat. No. 5,539,082). The phosphodiester bonds may be substituted with structures that are chiral and enantiomerically specific. Persons of ordinary skill in the art will be able to select other linkages for use in practice of the invention.

Oligonucleotides may also include species which include at least one modified nucleotide base. Thus, purines and pyrimidines other than those normally found in nature may be used. Similarly, modifications on the pentofuranosyl portion of the nucleotide subunits may also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙ NH₂ or O(CH₂)ₙ CH₃ where n is from 1 to about 10; C₁ to C₁₀ lower alkyl, substituted lower alkyl, alkaryl or aralkyl; Cl; Br; CN; CF₃ OCF₃; O, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic such as cyclobutyl or by another moiety which takes the place of the sugar.

In some embodiments, the antisense oligonucleotides in accordance with this invention may comprise from about 5 to about 100 nucleotide units. As will be appreciated, a nucleotide unit is a base-sugar combination (or a combination of analogous structures) suitably bound to an adjacent nucleotide unit through phosphodiester or other bonds forming a backbone structure.

In a further embodiment, expression of a PLA₂-encoding nucleic acid or fragment thereof may be inhibited or prevented using RNA interference (RNAi) technology, a type of post-transcriptional gene silencing. RNAi may be used to create a pseudo “knockout”, i.e. a system in which the expression of the product encoded by a gene or coding region of interest is reduced, resulting in an overall reduction of the activity of the encoded product in a system. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example Hammond et al. (2001), Sharp (2001), Caplen et al. (2001), Sedlak (2000) and published US patent applications 20020173478 (Gewirtz; published Nov. 21, 2002) and 20020132788 (Lewis et al.; published Nov. 7, 2002), all of which are herein incorporated by reference. Reagents and kits for performing RNAi are available commercially from for example Ambion Inc. (Austin, Tex., USA) and New England Biolabs Inc. (Beverly, Mass., USA).

The initial agent for RNAi in some systems is thought to be dsRNA molecule corresponding to a target nucleic acid. The dsRNA is then thought to be cleaved into short interfering RNAs (siRNAs) which are 21-23 nucleotides in length (19-21 bp duplexes, each with 2 nucleotide 3′ overhangs). The enzyme thought to effect this first cleavage step has been referred to as “Dicer” and is categorized as a member of the RNase III family of dsRNA-specific ribonucleases. Alternatively, RNAi may be effected via directly introducing into the cell, or generating within the cell by introducing into the cell a suitable precursor (e.g. vector encoding precursor(s), etc.) of such an siRNA or siRNA-like molecule. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

RNAi may be effected by the introduction of suitable in vitro synthesized siRNA or siRNA-like molecules into cells. RNAi may for example be performed using chemically-synthesized RNA (Brown et al., 2002). Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various vector-based methods are described in for example Brummelkamp et al. (2002), Lee et al. (2002), Miyagashi and Taira (2002), Paddison et al. (2002) Paul et al. (2002) Sui et al. (2002) and Yu et al. (2002). Various methods for introducing such vectors into cells, either in vitro or in vivo (e.g. gene therapy) are known in the art.

Accordingly, in an embodiment PLA₂ expression may be inhibited by introducing into or generating within a cell an siRNA or siRNA-like molecule corresponding to a PLA₂-encoding nucleic acid or fragment thereof, or to an nucleic acid homologous thereto. “siRNA-like molecule” refers to a nucleic acid molecule similar to an siRNA (e.g. in size and structure) and capable of eliciting siRNA activity, i.e. to effect the RNAi-mediated inhibition of expression. In various embodiments such a method may entail the direct administration of the siRNA or siRNA-like molecule into a cell, or use of the vector-based methods described above. In an embodiment, the siRNA or siRNA-like molecule is less than about 30 nucleotides in length. In a further embodiment, the siRNA or siRNA-like molecule is about 21-23 nucleotides in length. In an embodiment, siRNA or siRNA-like molecule comprises a 19-21 bp duplex portion, each strand having a 2 nucleotide 3′ overhang. In embodiments, the siRNA or siRNA-like molecule is substantially identical to a PLA₂-encoding nucleic acid or a fragment or variant (or a fragment of a variant) thereof. Such a variant is capable of encoding a protein having PLA₂-like activity. In embodiments, the sense strand of the siRNA or siRNA-like molecule is substantially identical to SEQ ID NOs:1 and/or 3, or a fragment thereof (RNA having U in place of T residues of the DNA sequence).

A number of PLA₂ inhibitors have been described. Such inhibitors include, but are not limited to arachidonic acid analogues such as the arachidonic acid analogues AACOCF₃ and MAFP described above, sialoglycolipids, proteoglycans and p-bromophenyl bromide as noted above, and certain benzenesulfonamide derivatives (Oinuma et al, 1991; European patent application No. 468 054). Further, bromoenol lactone and trifluoromethyl ketones (such as palmitoyl trifluoromethyl ketone, arachidonyl trifluoromethyl ketone) have been reported as inhibitors of Ca⁺⁺-independent PLA₂ (Ackermann et al, 1995) and cPLA₂ (Street et al, 1993) activity as well as bromophenacyl bromide. Accordingly, the invention further provides methods and uses of such compounds for the inhibition of inflammatory and/or demyelinating neural disease, such as MS and related neurodegenerative disease.

In another aspect, the invention relates to the use of a PLA₂ as a target in screening assays that may be used to identify compounds that are useful for the prevention or treatment of inflammatory and/or demyelinating neural disease, such as MS and related neurodegenerative disease.

In an embodiment, the invention further provides a method of identifying a compound for prevention and/or treatment of neural inflammatory and/or demyelinating disease, said method comprising (a) providing a test compound; and (b) determining whether PLA₂ activity is decreased in the presence of said test compound, wherein a decrease in said activity is indicative that said test compound may be used for treating a neural inflammatory and/or demyelinating disease.

In embodiments, such an assay may comprise the steps of
a) providing a test compound;
b) providing a source of enzymatically active PLA₂; and
c) determining PLA₂ activity in the presence versus the absence of the test compound, wherein a lower measured activity in the presence of the test compound indicates that the compound is an inhibitor of PLA₂ and is useful for the prevention and/or treatment of inflammatory and/or demyelinating neural disease, such as MS and related neurodegenerative disease.

In another aspect, the invention relates to the use of a PLA₂ as a target in screening assays that may be used to identify compounds that are useful for the prevention or treatment of inflammatory and/or demyelinating neural disease, such as MS and related neurodegenerative disease. In some embodiments, such an assay may comprise the steps of
a) providing a test compound;
b) providing a source of enzymatically active PLA₂;
c) providing a substrate for the PLA₂;
d) assaying the activity of the PLA₂ on the substrate in the presence of the compound, to identify compounds that inhibit the PLA₂, wherein said compound is useful for the prevention or treatment of inflammatory and/or demyelinating neural disease, such as MS and related neurodegenerative disease. In an embodiment the substrate is a phospholipid (e.g. phosphatidylcholine) comprising an arachidonoyl group at the sn-2 position.

The invention also relates to similar assays based on the detection on the expression of PLA₂ and PLA₂ protein levels, which can be detected for example by immunoassay methods or specific labeling methods, or via a reporter-based assay as noted below.

Such assays may further comprise the step of assaying the compounds for the reduction, abrogation or reversal of EAE symptoms. Such assays may be utilized to identify compounds that modulate expression of the PLA₂ gene, or compounds that modulate the activity of the expressed enzyme.

Screening assays of the invention may also be utilized to identify and/or characterize a compound for inhibiting demyelination. Therefore, the invention further provides a method for identifying and/or characterizing a compound for inhibiting demyelination, said method comprising assaying the activity of a PLA₂ in the presence of a test compound, to identify a compound that inhibits the PLA₂, wherein inhibition is indicative that the test compound may be useful for inhibiting demyelination. In an embodiment, the just noted method may further comprise assaying the compound for inhibition of demyelination.

The above-noted assays may be applied to a single test compound or to a plurality or “library” of such compounds (e.g. a combinatorial library). Any such compounds may be utilized as lead compounds and further modified to improve their therapeutic, prophylactic and/or pharmacological properties for the prevention and treatment of inflammatory and/or demyelinating neural disease, such as MS and related neurodegenerative disease.

Such assay systems may comprise a variety of means to enable and optimize useful assay conditions. Such means may include but are not limited to: suitable buffer solutions, for example, for the control of pH and ionic strength and to provide any necessary components for optimal PLA₂ activity and stability (e.g. protease inhibitors), temperature control means for optimal PLA₂ activity and or stability, and detection means to enable the detection of the PLA₂ reaction product, e.g. arachidonic acid and/or LPC. A variety of such detection means may be used, including but not limited to one or a combination of the following: radiolabelling (e.g. ³²P, ¹⁴C, ³H), antibody-based detection, fluorescence, chemiluminescence, spectroscopic methods (e.g. generation of a product with altered spectroscopic properties), various reporter enzymes or proteins (e.g. horseradish peroxidase, green fluorescent protein), specific binding reagents (e.g. biotin/(strept)avidin), and others.

The assay may be carried out in vitro utilizing a source of PLA₂ which may comprise naturally isolated or recombinantly produced PLA₂, in preparations ranging from crude to pure. Recombinant PLA₂ may be produced in a number of prokaryotic or eukaryotic expression systems, which are well known in the art (see for example U.S. Pat. No. 5,354,677 [Knopf et al., Oct. 11, 1994] for the recombinant expression of cPLA₂. Such assays may be performed in an array format. In certain embodiments, one or a plurality of the assay steps are automated.

A homolog, variant and/or fragment of PLA₂ which retains activity may also be used in the methods of the invention. Homologs include protein sequences, which are substantially identical to the amino acid sequence of a PLA₂, sharing significant structural and functional homology with a PLA₂. Variants include, but are not limited to, proteins or peptides, which differ from a PLA₂ by any modifications, and/or amino acid substitutions, deletions or additions. Modifications can occur anywhere including the polypeptide backbone, (i.e. the amino acid sequence), the amino acid side chains and the amino or carboxy termini. Such substitutions, deletions or additions may involve one or more amino acids. Fragments include a fragment or a portion of a PLA₂ or a fragment or a portion of a homolog or variant of a PLA₂.

The assay may in an embodiment be performed using an appropriate host cell comprising PLA₂ as a source of PLA₂. Such a host cell may be prepared by the introduction of DNA encoding PLA₂ into the host cell and providing conditions for the expression of PLA₂. Such host cells may be prokaryotic or eukaryotic, bacterial, yeast, amphibian or mammalian.

A number of methods for measuring PLA₂ activity may be utilized, such as those described by Reynolds et al. (1994) and Currie et al. (1994) or in U.S. Pat. No. 5,464,754 (Dennis et al., Nov. 7, 1995).

In another embodiment of the invention, a reporter assay-based method of selecting agents which modulate PLA₂ expression is provided. The method includes providing a cell comprising a nucleic acid sequence comprising a PLA₂ transcriptional regulatory sequence operably-linked to a suitable reporter gene. The cell is then exposed to the agent suspected of affecting PLA₂ expression (e.g. a test compound) and the transcription efficiency is measured by the activity of the reporter gene. The activity can then be compared to the activity of the reporter gene in cells unexposed to the agent in question. Suitable reporter genes include but are not limited to beta-D galactosidase, luciferase, chloramphenicol acetyltransferase and fluorescent green protein.

“Transcriptional regulatory sequence” is a generic term that refers to DNA sequences, such as initiation and termination signals, enhancers, and promoters, splicing signals, polyadenylation signals which induce or control transcription of protein coding sequences with which they are operably linked. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since enhancers generally function when separated from the promoters by several kilobases and intronic sequences may be of variable lengths, some polynucleotide elements may be operably-linked but not contiguous. In another embodiment, the construct may comprise an in frame fusion of a suitable reporter gene within the open reading frame of a PLA₂ gene. The reporter gene may be chosen as such to facilitate the detection of its expression, e.g. by the detection of the activity of its gene product. Such a reporter construct may be introduced into a suitable system capable of exhibiting a change in the level of expression of the reporter gene in response to exposure a suitable biological sample. Such an assay would also be adaptable to a possible large scale, high-throughput, automated format, and would allow more convenient detection due to the presence of its reporter component.

The above-described assay methods may further comprise determining whether any compounds so identified can be used for the prevention or treatment of inflammatory and/or demyelinating neural disease, such as MS and related neurodegenerative disease, such as examining their effect(s) on inflammatory cell influx and demyelination in lesions in the EAE animal model system.

In various embodiments, PLA₂ inhibitors, or pharmaceutically-acceptable salts thereof, may be used therapeutically in formulations or medicaments to prevent or treat inflammatory and/or demyelinating neural disease, such as MS and related neurodegenerative disease. The invention provides corresponding methods of medical treatment, in which a therapeutic dose of a PLA₂ inhibitor is administered in a pharmacologically acceptable formulation. Accordingly, the invention also provides therapeutic compositions comprising a PLA₂ inhibitor and a pharmacologically acceptable excipient or carrier. The therapeutic composition may be soluble in an aqueous solution at a physiologically acceptable pH.

In an embodiment, a compound of the invention (e.g. a PLA₂ inhibitor) is administered such that it comes into contact with CNS tissue, i.e. CNS neural cells. Such tissue includes brain and spinal cord (e.g. cervical, thoracic, or lumbar) tissue. As such, in embodiments a compound of the invention can be administered to treat CNS cells in vivo via direct intracranial injection or injection into the cerebrospinal fluid. Alternatively, the compound can be administered systemically (e.g. intravenously) and may come into contact with the affected CNS tissue via lesions (where the blood-brain barrier is compromised), or, in a further embodiment, may be in a form capable of crossing the blood-brain barrier and entering the CNS. Further, in an embodiment, a composition of the invention may be formulated for such CNS administration.

The invention provides pharmaceutical compositions (medicaments) containing (comprising) PLA₂ inhibitors. In one embodiment, such compositions include a PLA₂ inhibitor in a therapeutically or prophylactically effective amount sufficient to treat inflammatory and/or demyelinating neural disease, such as MS and related neurodegenerative disease.

The invention further provides a use of a PLA₂ inhibitor or a composition comprising a PLA₂ inhibitor for the prevention and/or treatment of inflammatory and/or demyelinating neural disease, or for the preparation of a medicament for the prevention and/or treatment of inflammatory and/or demyelinating neural disease.

A “therapeutically effective amount” refers to an amount effective, at dosages and for periods of time necessary, to achieve the desired therapeutic result, such as reduction of inflammatory and/or demyelinating neural disease, such as MS and related neurodegenerative disease progression. A therapeutically effective amount of PLA₂ inhibitor may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of inflammatory and/or demyelinating neural disease, such as MS and related neurodegenerative disease onset or progression. A prophylactically effective amount can be determined as described above for-the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the PLA₂ inhibitors can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. PLA₂ inhibitor) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a PLA₂ inhibitor may be formulated with one or more additional compounds that enhance the solubility of the PLA₂ inhibitor.

In accordance with another aspect of the invention, therapeutic compositions of the present invention, comprising a PLA₂ inhibitor, may be provided in containers or commercial packages which further comprise instructions for use of the PLA₂ inhibitor for the prevention and/or treatment of inflammatory and/or demyelinating neural disease, such as MS and related neurodegenerative disease.

Accordingly, the invention further provides a commercial package comprising a PLA₂ inhibitor or the above-mentioned composition together with instructions for the prevention and/or treatment of inflammatory and/or demyelinating neural disease, such as MS and related neurodegenerative disease.

The positive correlation of PLA₂ expression with EAE indicates that the assessment of the level of PLA₂ protein or a nucleic acid (e.g. an mRNA) encoding PLA₂ or PLA₂ enzyme activity is useful for the diagnosis or prognostication of inflammatory and/or demyelinating neural disease, such as MS and related neurodegenerative disease. PLA₂ mRNA levels may be assessed by methods known in the art such as Northern analysis or RT-PCR (see for example Sambrook et al (1989) Molecular Cloning: A Laboratory Manual (second edition), Cold Spring Harbor Laboratory Press, Cold Spring Harbor, N.Y., USA).

The level of PLA₂ protein or PLA₂ encoding mRNA or PLA₂ enzyme activity may be measured in a variety of tissues and body fluids including but not limited to blood, plasma, cerebrospinal fluid, neural cells (e.g. astrocytes) endothelial cells and immune cells (e.g. macrophages, leukocytes and lymphocytes).

In an embodiment, the level of PLA₂ protein or PLA₂ encoding mRNA or PLA₂ enzyme activity measured in an animal to be tested may be compared to an established standard of PLA₂ protein or PLA₂ encoding mRNA or PLA₂ enzyme activity.

In an embodiment, the level of PLA₂ protein or PLA₂ encoding mRNA or PLA₂ enzyme activity measured in an animal to be tested may be compared to a corresponding level of PLA₂ protein or PLA₂ encoding mRNA or PLA₂ enzyme activity measured in tissue or body fluid of a control animal. In an embodiment, the control animal is an age- and/or weight-matched animal.

In an embodiment, the level of PLA₂ protein or PLA₂ encoding mRNA or PLA₂ enzyme activity measured in an animal to be tested may be compared to a corresponding level of PLA₂ protein or PLA₂ encoding mRNA or PLA₂ enzyme activity measured in tissue or body fluid of the same animal at an earlier time, and such a method is used to prognosticate inflammatory and/or demyelinating neural disease, such as MS and related neurodegenerative disease.

In an embodiment, the diagnostic/prognostic method is used in conjunction with methods for identifying patients suspected of suffering from neural inflammatory and/or demyelating disease, such as MS. For example the diagnostic/prognostic method of the invention may further comprise the step of identifying a subject exhibiting symptoms associated with neural inflammatory and/or demyelating disease, such as MS. Such symptoms include but are not limited to optic neuritis, weakness, e.g. in the limbs, tingling or similar sensation, and loss or decrease (e.g. a transient loss or decrease) of motor control (e.g. in the limbs).

According to a further aspect of the present invention, a commercial package is provided for the diagnosis or prognostication of inflammatory and/or demyelinating neural disease, such as MS and related neurodegenerative disease in an animal. The commercial package comprising means for the assessment of the level of PLA₂ protein or PLA₂ encoding mRNA or PLA₂ enzyme activity in a tissue or body fluid of the animal together with instructions for the diagnosis or prognostication of inflammatory and/or demyelinating neural disease, such as MS and related neurodegenerative disease.

The invention further relates to the use of anti-PLA₂ antibodies for prophylactic, therapeutic, diagnostic and/or prognostic uses. With regard to therapeutic uses, an anti-PLA₂ antibody may be used which is capable of modulating (e.g. inhibiting) the binding and/or catalytic activity of a PLA₂. With regard to diagnostic and prognostic uses, an anti-PLA₂ antibody may be used for detecting PLA₂ and, in embodiments, quantifying the level thereof, in a sample, such as a tissue or body fluid and lymphocytes. Such detection may further be used for imaging methods.

Some anti-PLA₂ antibodies have already been described, such as the anti-cPLA₂ utilized in the Examples below . To prepare such antibodies, a PLA₂ or fragment/homolog/variant thereof may be used to immunize a small mammal, e.g., a mouse or a rabbit, in order to raise antibodies which recognize a PLA₂. An anti-PLA₂ antibody may be either polyclonal or monoclonal. Methods to produce polyclonal or monoclonal antibodies are well known in the art. For a review, see Harlow and Lane (1988) and Yelton et al. (1981), both of which are herein incorporated by reference. For monoclonal antibodies, see Kohler and Milstein (1975), herein incorporated by reference.

Antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monoclonal antibodies may also be in the form of immunoglobulin fragments, e.g., F(ab)′₂, Fab or Fab′ fragments. The antibodies may be of any isotype, e.g., IgG or IgA, and polyclonal antibodies are of a single isotype or a mixture of isotypes.

Anti-PLA₂ antibodies may be produced and identified using standard immunological assays, e.g., Western blot analysis, dot blot assay, or ELISA (see, e.g., Coligan et al. (1994), herein incorporated by reference). The antibodies are used in diagnostic methods to detect the presence of a PLA₂ in a sample, such as a biological sample.

Accordingly, a further aspect of the invention provides a method for assessing an inflammatory and/or demyelinating neural disease, such as MS and related neurodegenerative disease, in an animal, based on detecting the presence of a PLA₂ in a biological sample obtained from the animal, by contacting the biological sample with an antibody capable of recognizing a PLA₂, such that an immune complex is formed, and by detecting such complex to indicate the presence of PLA₂ in the sample.

Those skilled in the art will readily understand that the immune complex is formed between a component of the sample and the antibody, and that any unbound material is removed prior to detecting the complex. It is understood that such an antibody is used for screening a sample, such as plasma, leukocytes, lymphocytes, macrophages, cerebrospinal fluid, urine, saliva, neural cells and endo- or epi-thelia for the presence of PLA₂.

For diagnostic applications, the reagent (i.e., an anti-PLA₂ antibody) is either in a free state or immobilized on a solid support, such as a tube, a bead, or any other conventional support used in the field. Immobilization is achieved using direct or indirect means. Direct means include passive adsorption (non-covalent binding) or covalent binding between the support and the reagent. By “indirect means” is meant that an anti-reagent compound that interacts with a reagent is first attached to the solid support. Indirect means may also employ a ligand-receptor system, for example, where a molecule such as a vitamin is grafted onto the reagent and the corresponding receptor immobilized on the solid phase. This is illustrated by the biotin-(strept)avidin system. Alternatively, a peptide tail is added chemically or by genetic engineering to the reagent and the grafted or fused product immobilized by passive adsorption or covalent linkage of the peptide tail.

Such diagnostic agents may be included in a commercial package or kit which also comprises instructions for use. The reagent is labeled with a detection means which allows for the detection of the reagent when it is bound to its target. The detection means may be a fluorescent agent such as fluorescein isocyanate or fluorescein isothiocyanate, or an enzyme such as horseradish peroxidase or luciferase or alkaline phosphatase, or a radioactive element such as ¹²⁵I or ⁵¹Cr.

A further aspect of the present invention is a diagnostic imaging method, which comprises introducing into a biological system, an anti-PLA₂ antibody, which is used in conjunction with an appropriate detection system to identify areas where PLA₂ is present or absent.

The invention further relates to the role of PLA₂ in a variety of in vitro and in vivo inflammatory and/or demyelinating neural disease systems, such as MS and related neurodegenerative disease model systems, such as the EAE model system, and the use of such systems for inflammatory and/or demyelinating neural disease, such as MS and related neurodegenerative disease research. Accordingly, the invention provides a variety of in vitro and in vivo model systems for the study of the mechanisms of the development and progression of inflammatory and/or demyelinating neural disease, such as MS and related neurodegenerative disease, and for the development and characterization of materials and methods for the prevention, treatment, and/or diagnosis of inflammatory and/or demyelinating neural disease, such as MS and related neurodegenerative disease. In an embodiment, such a system comprises a mutation or disruption in a PLA₂ gene or other means of PLA₂ inactivation. In embodiments, the PLA₂ gene encodes a PLA₂ which is cytosolic or secreted, calcium dependent or independent. In an embodiment, the PLA₂ is a cytosolic PLA₂. In an embodiment, both copies of the gene are mutated or disrupted. The system may comprise a transgenic non-human mammal, such as a rodent, such as a mouse.

Applicants have determined that immune cell influx and demyelination at neural lesions correlate with PLA₂ expression and activity. Accordingly, the invention further provides a method of inhibiting immune cell influx and demyelination at neural lesions in an biological system, via inhibiting the activity and/or expression of a PLA₂ in said system. The invention further provides a use of a PLA₂ inhibitor for the inhibition of immune cell influx and/or demyelination at neural lesions in a biological system, or for the preparation of a medicament for the inhibition of immune cell influx and/or demyelination at neural lesions in a biological system. The invention further provides a method of assessing immune cell influx and/or demyelination at neural lesions in a biological system, the method comprising:
(a) determining a test level of PLA₂ protein or PLA₂ encoding mRNA or PLA₂ enzyme activity in said system; and
(b) comparing said test level of PLA₂ protein or PLA₂ encoding mRNA or PLA₂ activity to an established standard;
or to a corresponding level of PLA₂ protein or PLA₂ encoding mRNA or PLA₂ enzyme activity in a control system;
or to a corresponding level of PLA₂ protein or PLA₂ encoding mRNA or PLA₂ enzymatic activity determined in said system at an earlier time;
   wherein an increase in said test level is indicative of immune cell influx and/or demyelination at neural lesions.

The invention further provides a commercial package comprising a PLA₂ inhibitor together with instructions for inhibiting immune cell influx and/or demyelination at neural lesions. The invention further provides a commercial package comprising means for the assessment of the level of PLA₂ or PLA₂ encoding mRNA or PLA₂ enzyme activity in a biological system together with instructions for assessing immune cell influx and/or demyelination at neural lesions in biological system.

In embodiments, the above noted biological system is a mammal, in a further embodiment, a human.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

### Materials and Methods

Generation of EAE: EAE was induced in female C57BL/6 mice (18-20 g) by subcutaneous injections of 50 μg of myelin oligodendrocyte glycoprotein (MOG₃₅₋₅₅-MEVGWYRSPFSRVVHLYRNGK [SEQ ID NO:5]) (Sheldon Biotechnology Centre, McGill University) in Complete Freund's Adjuvant (Incomplete Freund's adjuvant containing 1 mg heat inactivated Mycobacterium tuberculosis (Difco Labs)). An intravenous injection of 200 ng of pertussis toxin (List Biologicals) was also administered on days 0 and 2. The mice were monitored clinically for EAE symptoms daily using the following 5-point scale:
Grade 0=normal (no clinical signs).
Grade 1=flaccid tail.
Grade 2=flaccid tail and mild hindlimb weakness (fast righting after mice are placed on their backs).
Grade 3=flaccid tail and severe hindlimb weakness (slow righting after mice are placed on their backs).
Grade 4=flaccid tail and hindlimb paralysis.
Grade 5=flaccid tail, hindlimb paralysis plus forelimb weakness/moribund.

Immunohistochemistry: The mice at different clinical grades were deeply anesthetized and perfused via the heart with 0.1 M phosphate buffer (pH 7.2) followed by perfusion with 4% paraformaldehyde in 0.1 M phosphate buffer (pH 7.2). The spinal cords of the mice were post-fixed for an hour in the same fixative, and then cryoprotected overnight in 30% sucrose in phosphate buffered saline (PBS). Cryostat sections (14 μm) of cross sections of the cervical, thoracic and lumbar spinal cord were incubated in 0.1% H₂O₂ to remove endogenous peroxidases, and then blocked in 0.1% Triton-X 100 and 2% normal goat serum for 5 hours. Tissues were then incubated with an antibody against cPLA₂ (polyclonal rabbit anti-cPLA₂-Santa Cruz Biotech) overnight. Tissue sections were then incubated with a biotinylated goat anti-rabbit antibody and then washed and incubated with the avidin-biotin complex conjugated to horseradish peroxidase (Vectastain kit). The staining was visualized using diaminodibenzidine (Sigma) using protocols described previously (Ousman and Pavid, 2000). Sections were counterstained with 3% methyl green, and then dehydrated in ethanol. The slides were cover slipped in Permount.

Double Immunofluorescence: Cryostat sections of tissue obtained by perfusion as described above were blocked in 0.1% Triton-X 100 and 2% normal goat serum and then incubated overnight with an antibody against cPLA₂ (same as that described above) combined with either antibodies specific for astrocytes (mouse anti-GFAP-Sigma), endothelial cells (rat anti-CD34-BD PharMingen), T cells (rat anti CD4-PharMingen), or macrophages (monoclonal antibody Mac-1). Tissue sections were then washed and incubated with a biotinylated goat anti rabbit secondary antibody combined with the appropriate goat anti-rat/mouse rhodamine-conjugated secondary antibody. Tissue sections were then washed and incubated with fluorestein-conjugated steptavidin. The slides were washed and cover slipped in phenylenediamine containing mounting medium.

Quantification: Counts were done using an ocular grid. For the immunoperoxidase stained sections, two cPLA₂⁺ cell types were counted: round cells (immune cells in the infiltrate at and near EAE lesions) and elongated cells (endothelial cells). Three levels of the spinal cord (cervical, thoracic and lumbar) were quantified for 3 animals in each grade (1-5). Counts were made on three sections at least 45 μm apart. The positive cells were taken as a percentage per lesion.

For the immunofluorescence stained sections, the number of cPLA₂⁺ cells, Mac-1⁺ cells and CD4⁺ T cells were quantified in 5 random lesions in the three levels of the spinal cord from 3 animals in each grade (1-5).

Mice given delayed treatment and their controls were analyzed on day 60. Mice were perfused with 4% paraformaldehyde and cryostat cross sections (12 μm) of the spinal cord were stained with hematoxylin and eosin (H&E). Inflammatory lesions were quantified by counting the number of lesions, the number of inflammatory cells and the area of the infiltrates per section (3 sections per animal and 3 animals per group).

Results are presented as the mean number of cells per group±standard error of the mean (s.e.m.). The statistical significance (P<0.05) between the various grades was determined by using a two-sample Student's t-test.

Electron microscopy: On day 60 mice were perfused with 0.5% paraformaldehyde and 2.5% glutaraldehyde in 0.1M phosphate buffer. Tissues were then post-fixed in 2% osmium tetroxide and processed and embedded in Epon as described previously (Ousman and David, 2000). Thin sections on Collodion-coated slot grids were stained with lead citrate and examined with a Philips CM10 electron microscope.

Treatment of EAE-induced mice with cPLA₂ inhibitors: EAE was induced in C57BL/6 mice as mentioned above. At days 0 and 2 a 50 μl intravenous injection of either 2 mM or 4 mM arachidonyl trifluoromethyl ketone (AACOCF₃-Cayman Chemicals) diluted in 1% DMSO buffer was administered. This was followed on alternated days by intraperitoneal injections of 200 μl of the same inhibitor at 2 or 4 mM concentrations until day 24. The mice were scored clinically based on the scoring system described above. Monitoring was done in a blinded fashion so that the person doing the scoring was unaware of the treatment groups.

### Example 2

### Expression of CPLA₂ in the Spinal Cord in EAE

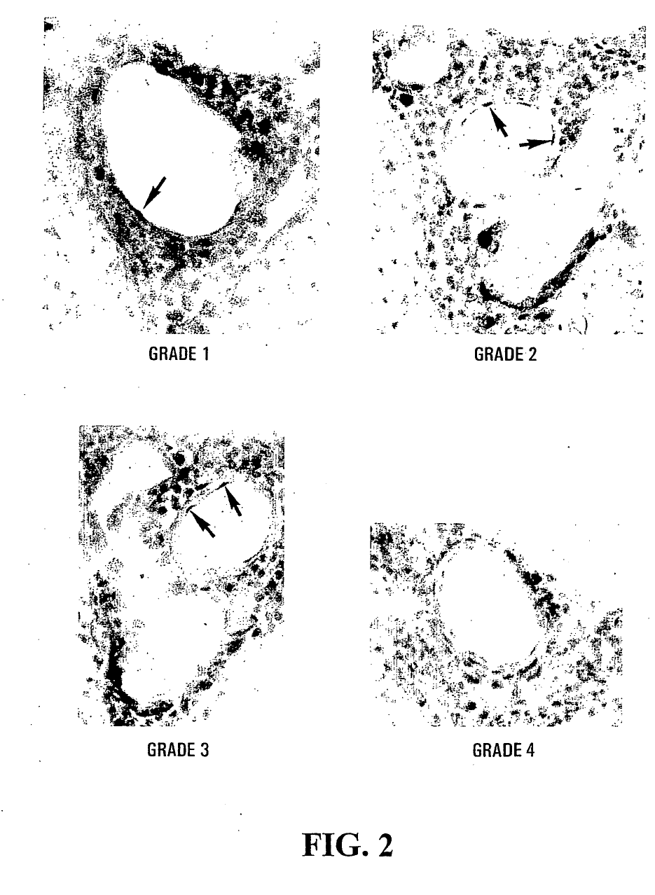

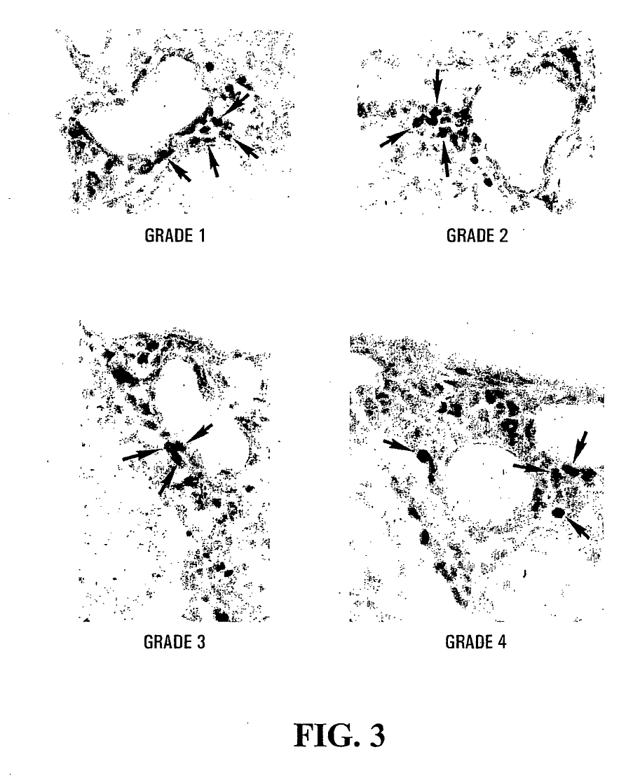

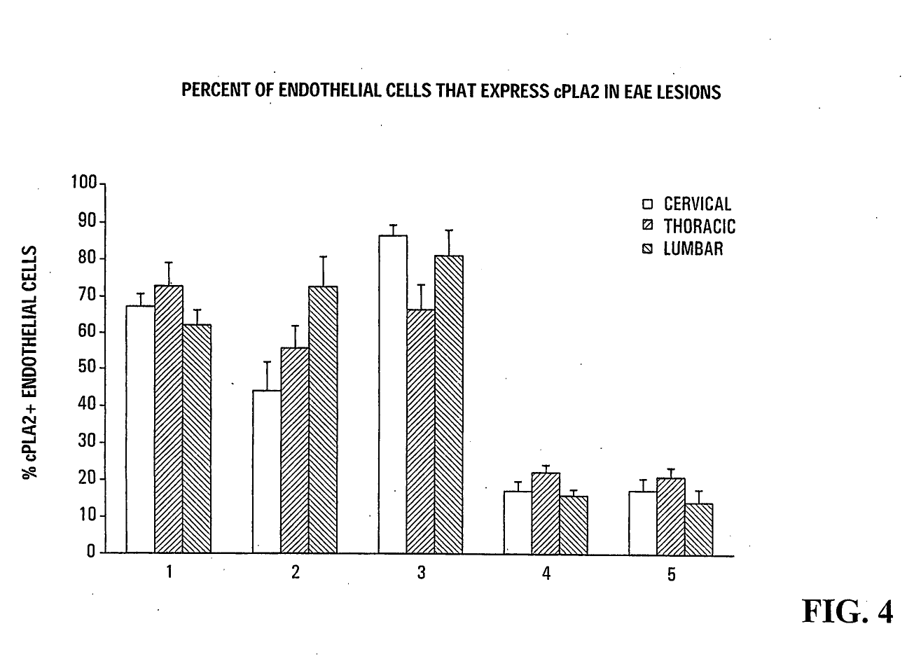

The expression of cPLA₂ in EAE was assessed in the C57BL/6 mouse strain, which has a naturally occurring null mutation for sPLA₂ group IIA (32), the major form of sPLA₂ in the CNS. Therefore, if PLA₂ plays a role in the onset of MOG-induced EAE in C57BL/6 mice, it has to be mediated mainly by cPLA₂. By the immunoperoxidase technique, increased expression of cPLA₂ was observed at the site of EAE lesions in the spinal cord. The labeling occurred in endothelial cells (FIG. 2), as well as immune cells in the CNS inflammatory infiltrates (FIG. 3). The percentage of cPLA₂⁺ endothelial cells ranged from 70% to 85% between clinical grades 1-3, and decreased to about 20% at clinical grades 4 and 5 (FIG. 4).

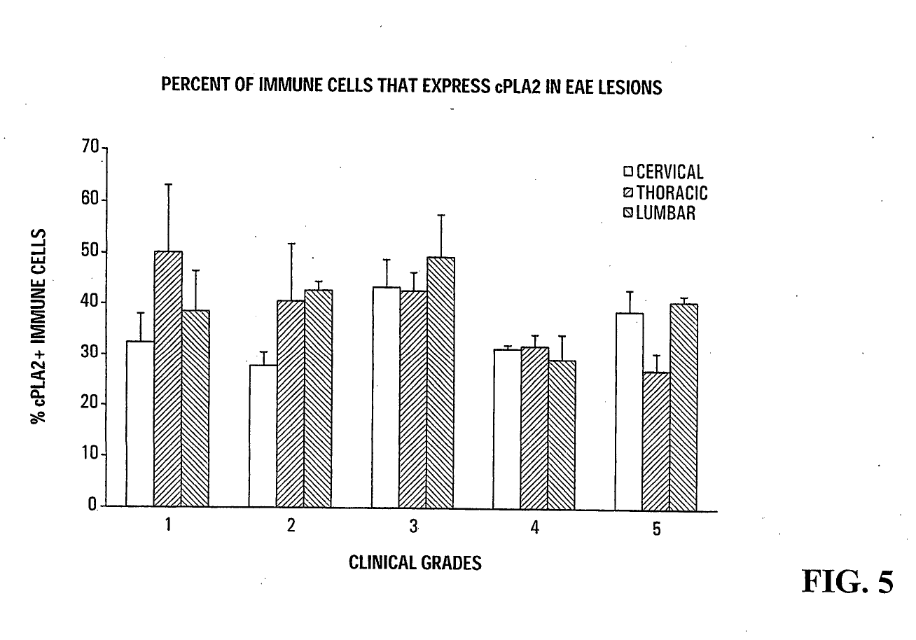

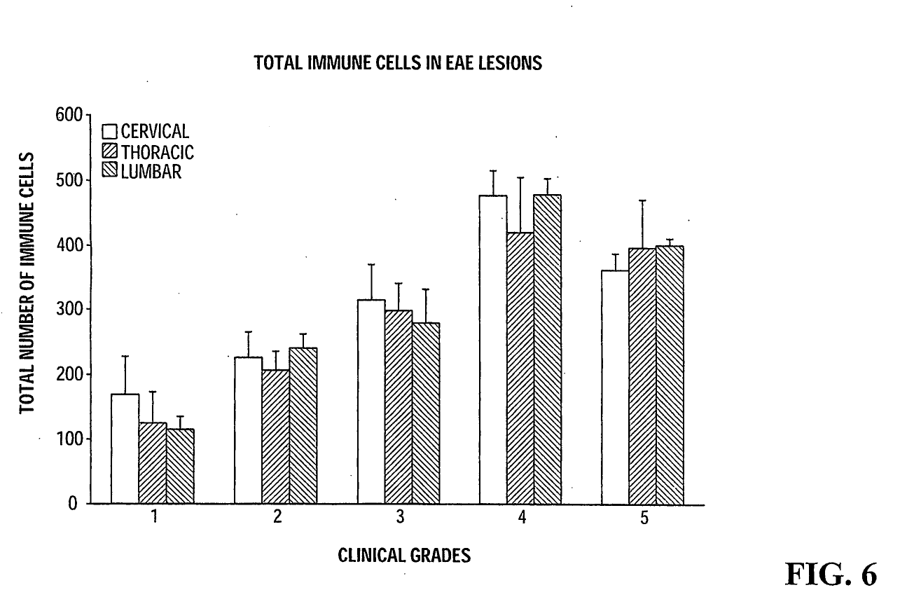

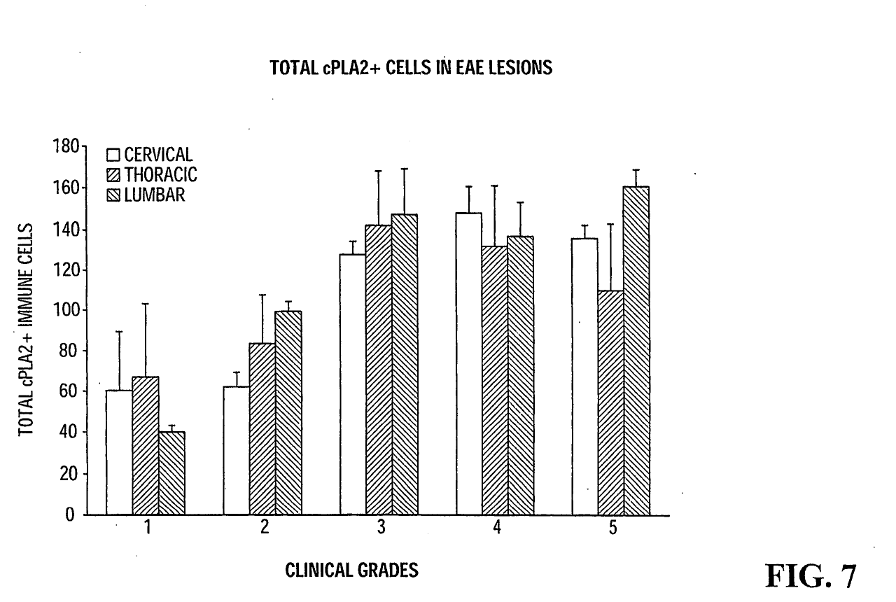

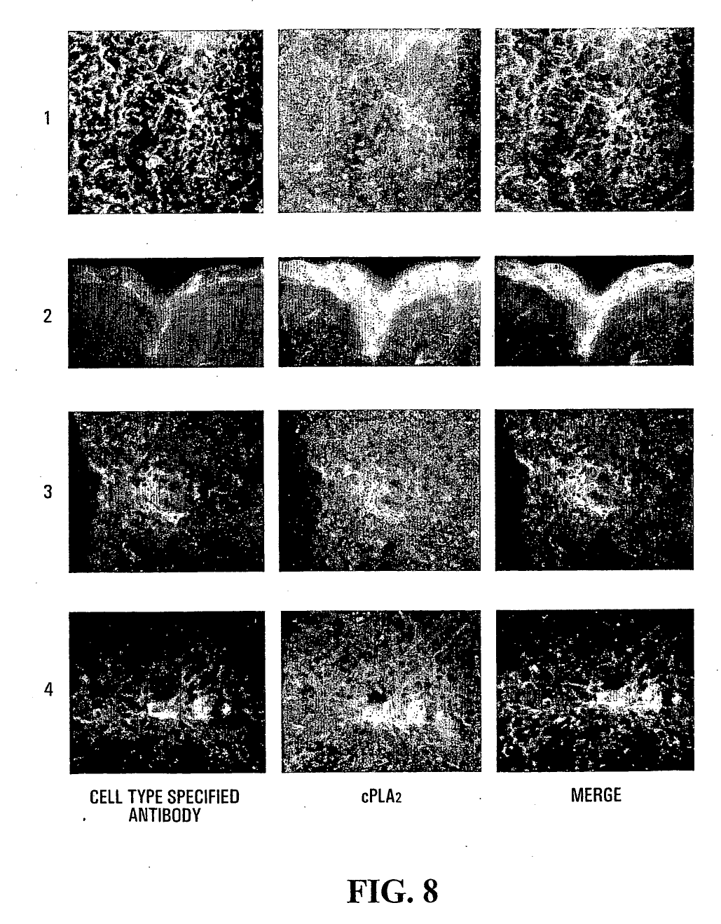

The percentage of cPLA₂⁺ round cells in the immune cell infiltrates in EAE lesions remained at around 30%-50% in all clinical grades (FIG. 5). However, since the total number of cells in the infiltrates increases with increasing clinical grades and with higher inflammatory scores (FIG. 6), the total number of cPLA₂ cells in the spinal cord increased with increasing severity of the disease (FIG. 7). Double-immunofluorescence labeling studies indicate that T cells, macrophages and astrocytes near EAE lesions express cPLA₂ (FIG. 8). These results show that the highest number of cPLA₂⁺ endothelial cells are seen at grades 1-3 which precedes the period of highest influx of inflammatory cells at grade 4.

### Example 3

### Blocking with a cPLA₂ Inhibitor Prevents the Onset of EAE

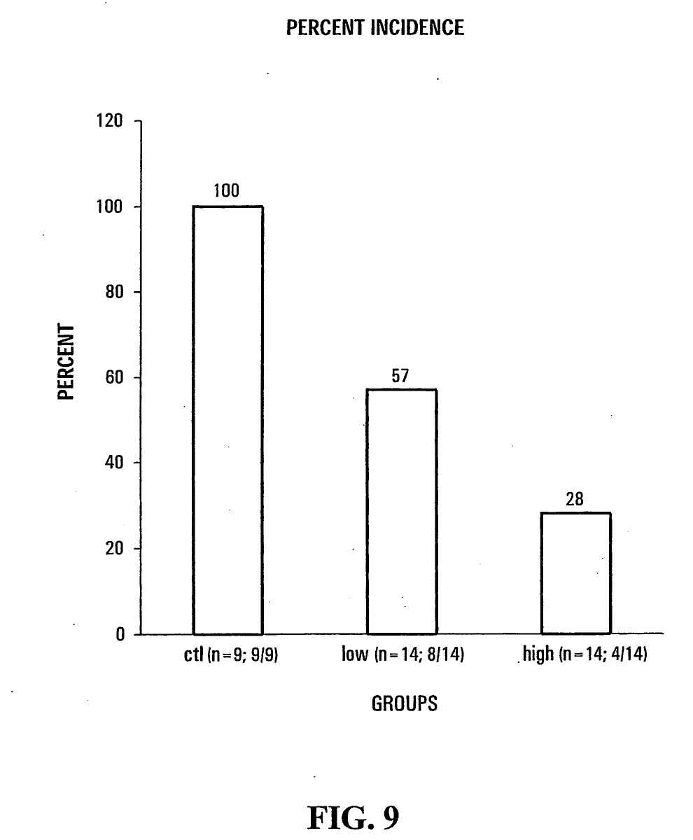

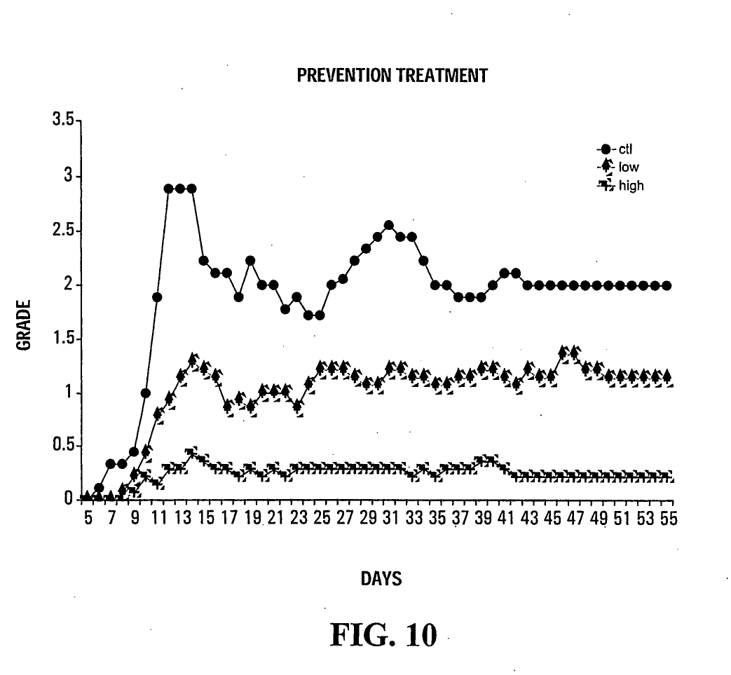

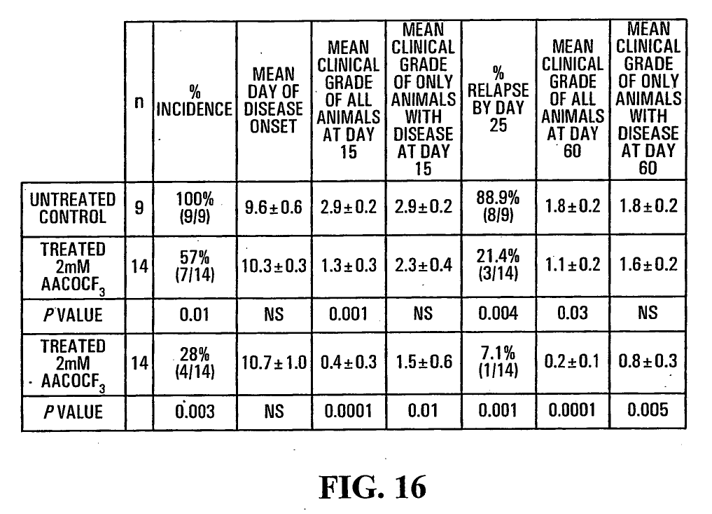

To assess if cPLA₂ is important for the onset of EAE we blocked it using a chemical inhibitor. C57BL/6 mice were treated with the cPLA₂ inhibitor AACOCF₃ on the day of immunization and on day 2 with 50 μl of 2 or 4 mM AACOCF₃ intravenously, followed by intraperitoneal injections of the inhibitor (200 μl at 2 or 4 mM) on alternate days until day 24. Mice were monitored clinically using the scoring scale described above. Treatment with the inhibitor resulted in a remarkable reduction in the onset and progression of EAE. 100% of the vehicle-treated control mice got EAE, while 57% of the 2 mM treated and only 28% of the 4 mM treated groups got EAE (FIG. 9; FIG. 16). The progression of the disease was also markedly reduced as shown in FIG. 10. Vehicle-treated controls reached an average maximum clinical score of 2.9 at 12-14 days, while the 2 mM and 4 mM treated groups reached scores of 1.5 and 0.4, respectively (FIG. 10; FIG. 16). Unlike the controls, which relapsed into a second paralytic episode between days 25-34, mice treated with 4 mM AACOCF₃ remained unaffected (FIG. 10; FIG. 16). The analysis was carried out blind, so that the person doing the clinical scoring was unaware of the treatment groups. The treatment is well tolerated in that the animals do not show any side-effects. The body weight and food-intake of treated mice were unaffected compared to controls at 35 days after induction of EAE. These results provide very strong evidence that blocking PLA₂ has a profound effect in the prevention of EAE.

### Example 4

### Delayed Treatment of EAE-Induced Mice with a cPLA₂ Inhibitor

Materials and methods:

EAE was induced in C57BL/6 mice as described above. A 50 μl intravenous injection of either 4 mM AACOCF₃ diluted in 1% DMSO containing buffer or vehicle (1% DMSO containing buffer) was administered on days 14, 16, 18 and 20 after induction of EAE, when animals began to remit. The mice were scored clinically in a blinded fashion as mentioned above.

Results:

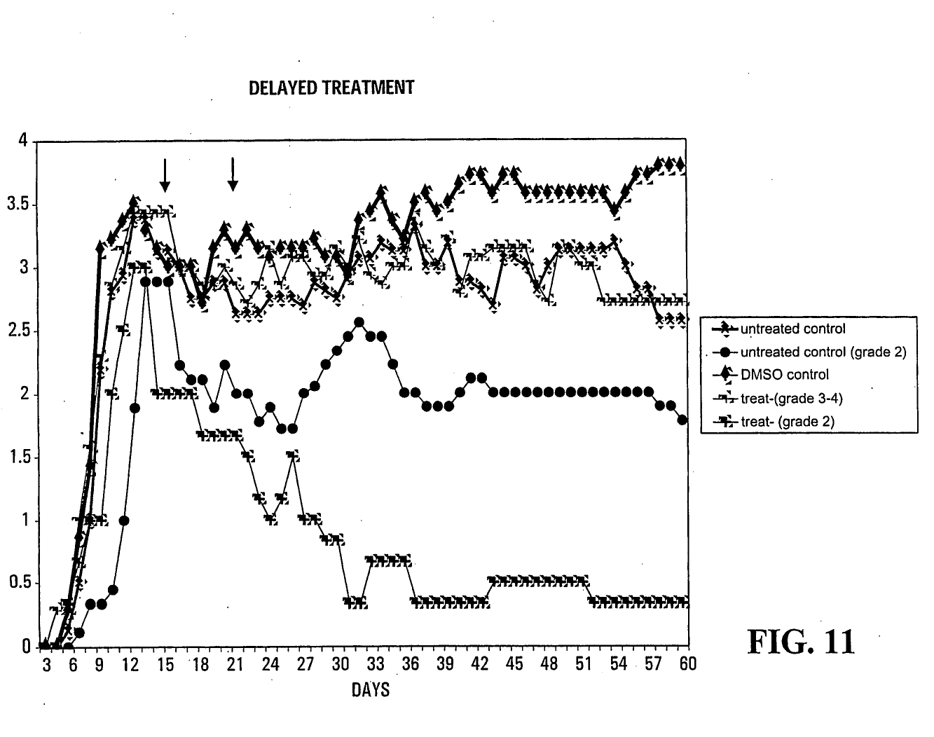

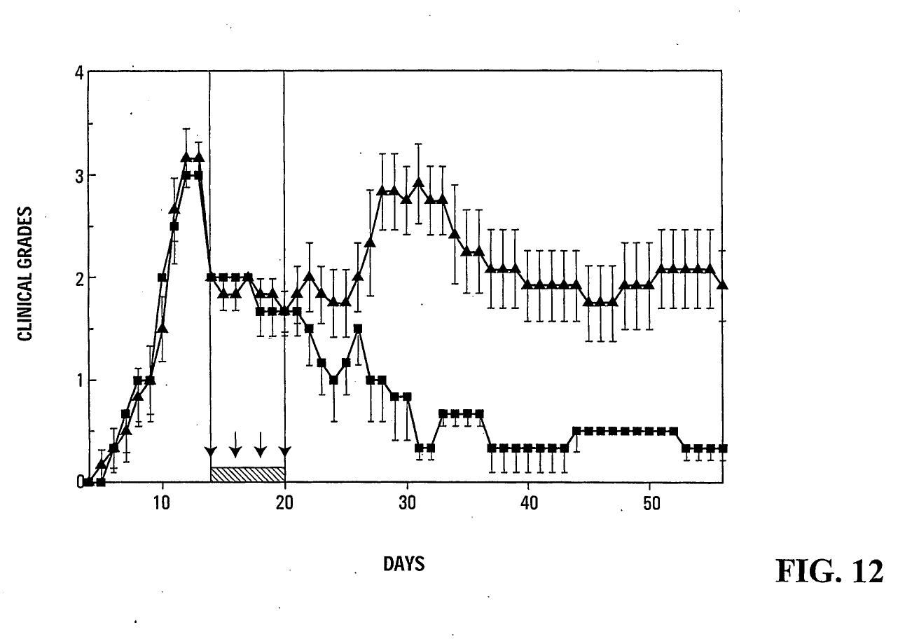

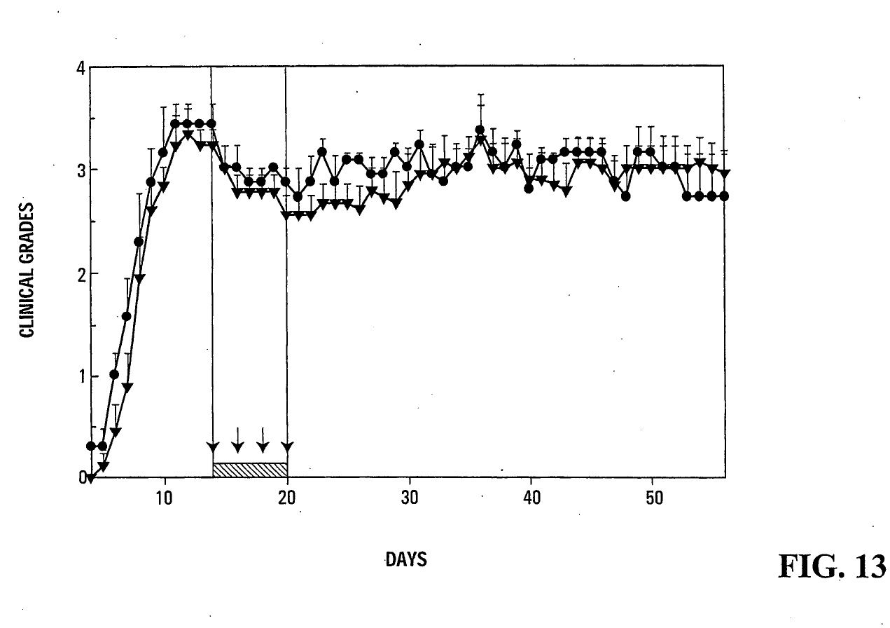

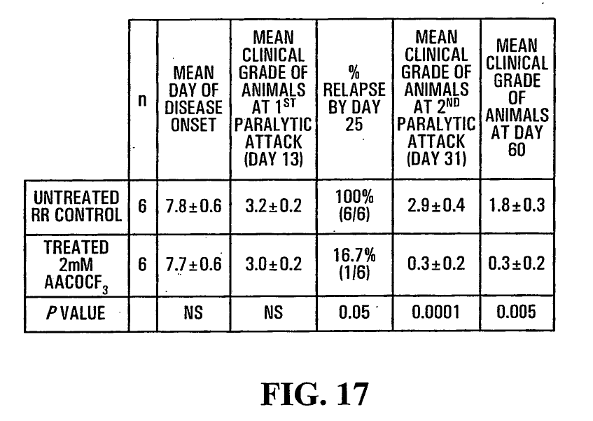

Blocking with cPLA₂ inhibitor prevents further relapse: To assess if blocking cPLA₂ is also effective in improving symptoms of EAE after the disease is well established, C57BL/6 mice that were induced with EAE were given a delayed treatment with 4 mM of AACOCF₃. All animals reached a score of grade 3 or above by day 13. Treatments for all mice were begun on day 14 when remissions were first seen. As shown in FIG. 11, the animals were given a one-week treatment (indicated by arrows in FIG. 11) and were monitored in a blind fashion using the clinical scoring scale described above. As shown in FIGS. 12 and 13, four 50 μl intravenous injections of the inhibitor were given during a one-week period. The animals were divided into two groups based on whether they had a clinical score of grade 3 or above or a score of grade of 2 or below on the first day of treatment. Both treated groups showed a similar clinical course as compared to their respective untreated controls during the one-week treatment period (FIGS. 11 to 13). The group that remitted to a clinical score of 2 or below on the first day of treatment showed a remarkable reduction in the progression of the disease (FIGS. 11 and 12). Although these animals had initially peaked to a mean clinical score of grade 3 prior to treatment, they progressively dropped down to a mean grade of 0.3 and remained virtually symptom free for up to day 60 (FIG. 12, FIG. 17), the maximum period studied. Their progression into a second relapse was prevented. In contrast, their matching untreated controls that also remitted to a clinical score of grade 2 or below on day 14 and which had initially peaked to a mean grade of 3.2, progressed into a second paralytic episode (grade 2.9) between days 25 and 37. These mice, which display a relapsing-remitting form of the disease, remained thereafter at a mean clinical score of 1.8 until day 60 (FIG. 12, FIG. 17). On the other hand, animals that had a clinical score of grade 3 or above on the first day of treatment showed a chronic form of the disease and were unaffected by the treatment regime, not differing significantly from the control group (FIG. 13). These groups reached a peak mean clinical score of 3.4 (FIG. 13).

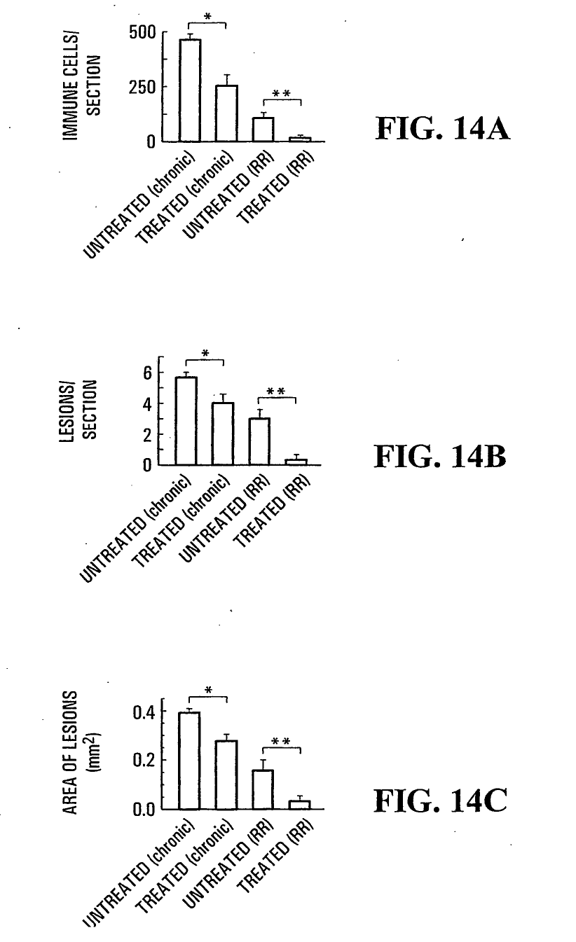

Although the mice that displayed a chronic course did not show clinical improvement with the inhibitor treatment, we assessed whether the treatment had any effect on inflammation in the spinal cord. Immune cell infiltration into the spinal cord was estimated by cell counts on hematoxylin and eosin (H&E) stained tissue sections of the spinal cord obtained at day 60 (n=3 in each group). The untreated chronic control group (grade 2.9 on day 60) had an average of about 460 immune cells in the lumbar spinal cord, with about 6 lesions per section, and a lesion area of about 0.39 mm² (FIG. 14a, b and c). In contrast, the treated chronic group that did not show clinical improvement (grade 2.8 on day 60), showed a reduced inflammatory burden. These treated mice had only about 250 immune cells in EAE lesions in the lumbar spinal cord, with an average of 4 lesions per section, and a lesion area of 0.28 mm² (FIG. 14a, b and c). Similar results were also seen in the cervical and thoracic regions. These results show that the PLA₂ inhibitor treatment does reduce the inflammatory response in the chronic form of EAE. Furthermore, there was a marked reduction in the lesion burden and immune cell infiltration in the other treated group, which had remitted to a grade of 2 or less and which responded well to inhibitor treatment (FIG. 14a, b and c). These results show that blocking cPLA₂ reduces the inflammatory burden in mice with EAE.

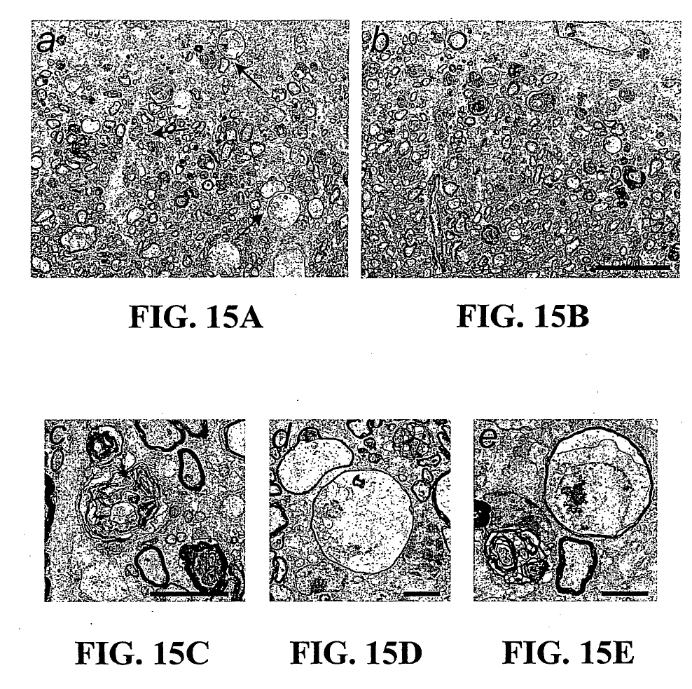

Although cPLA₂ inhibitor treatment was able to partially block inflammation in the chronic group, this reduction in inflammation did not lead to an improvement in the clinical score suggesting that other factors play a role. We therefore examined Epon embedded sections of the spinal cord by electron microscopy to assess whether there was axonal damage. Treated animals, which followed a chronic clinical course and their untreated controls both showed a great deal of axonal damage in areas near EAE lesions (FIG. 15a-e). This axonal loss may account for the permanent clinical deficits seen in these groups. In contrast to these chronic groups, the control group with the relapsing-remitting course (mean clinical score of 1.8 on day 60) showed fewer damaged axons, while the companion treated group, which improved with treatment (mean clinical score of 0.3 on day 60) showed virtually no damaged axons (data not shown). These data indicate that the failure of mice with the more severe form of EAE to improve with the inhibitor treatment may be due to permanent axonal damage. This axonal damage may be prevented if the treatment is started at an earlier time, i.e., before the peak of the first attack of EAE symptoms.

All references cited herein or in the references section below are herein incorporated by reference.

### REFERENCES

Ackermann et al. (1995) J. Biol. Chem. 270:445-50.
Adelmann, M. et al. (1995) J. Neuroimmunol. 63: 17-27.
Allen, I. et al. (1993) J. Neuropathol. Exp. Neurol. 52: 95-105.
Andersson, P. B. et al. (1998) J. Neurol. Sci. 160(1):16-25.
Arbibe, L. et al. (1997) J. Immunol. 159: 391-400.
Balsinde, J. et al. (1999) Annu. Rev. Pharmacol. Toxicol. 39: 175-189.
Bansil, S. et al. (1995) Ann. Neurol. 37 (S1):S87-S101.
Bauer, J. et al. (1995) Glia. 4: 437-46.
Bignami, A. et al. (1969) Brain Res. 13: 444-461.
Bjartmar, C. et al. (2001) Curr. Opin. Neurol. 14: 271-8.
Bonventre, J. V. et al. (1997) Reduced fertility and postischemic brain injury in mice deficient in cytosolic phospholipase A₂.
Brenner, T. et al. (1992) J. Neuroimmunol. 40(2-3): 273-9.
Brenner, T. et al. (1997) J. Immunol. 158(6): 2940-6.
Brenner, T. et al. (1998) Exp. Neurol. 154(2): 489-98.
Bruck, W. et al. (1994) Ann. Neurol. 35: 65-73.
Chang, H. W. et al. (1987) J. Biochem. 102: 147-154.
Chen, S. et al. (1993) J. Comp. Neurol. 333:449-454.
Currie et al. (1994) J. Biochem. 304: 923-8.
Dennis, E. (1994) J. Biol. Chem. 269: 13057-13060.
Dennis, E. A. (2000) Am. J. Respir. Crit. Care Med. 161: S32-S35.
Dinter, H. et al. (2000) J. Neuroimmunol. 108(1-2): 136-46.
Dore-Duffy, P. et. al. (1986) Neurology 36: 1587-1590.
Dore-Duffy, P. et al. (1991) Neurology 41: 322-324.
Ebers, G. C. (1996) Curr. Opinion Neurology 9: 155-158.
Emerson, M. R. et al. (2001) J. Neuroimmunol. 119(2): 205-13.
Ewing, C. et al. (1998) Immunology & Cell Biology 76: 47-54.
Farooqui, A. A. et al. (1997) J. Neurochem. 69: 889-901.
Fife, B. T. et al. (2001) J. Immunol. 166(12): 7617-24.
Forst, S. et al. (1986) Biochemistry 25: 8381-8385.
Fretland, D. J. (1992) Prostaglandins Leukot. Essent. Fatty Acids 45(4): 249-57.
Fujimori, Y. et al. (1992) J. Biochem. 111: 54-60
Gallai, V. et al. (1995) J. Neuroimmunol. 56(2): 143-53.
Glaser, K. B. et al. (1993) Trends in Pharmacol. 14: 92-98.
Goppelt-Struebe, M. et al. (1992) Biochem. Biophys. Acta. 1127: 163-167.
Goverman, J. et al. (1993) Cell 72: 551-560.
Hall, S. M. (1989) Neuropathol. & Appl. Neurobiol. 15: 513-530.
Hall, S. M. (1993) J. Neurocytol. 22: 480-490.
Harlow, E. et al. (1988) Antibodies: A Laboratory Manual, Cold Spring Harbor Laboratory Press, Cold Spring Harbor, N.Y.
Hauser, S. L. et al. (1990) Neurology 40: 1735-1739.
Hirashima, Y. et al. (1992) J. Neurochem. 59: 708-714.
Hofman, F. M. et al. (1986) J. Immunol. 136: 3239-3245.
Hofman, F. M. et al. (1989) J. Exp. Med. 170: 607-612.
Huitinga, I. et al. (1990) J. Exp. Med. 172(4): 1025-33.
Hulkower, K. I. et al. (1992) Biochem. Biophys. Res. Commun. 184: 712-718.
Huterer, S. J. et al. (1995) Neurochem. Res. 20: 1335-1343.
Ishizaki, J. et al. (1989) Biochem. Biophys. Res. Commun. 162: 1030-1036.
Issazadeh, S. et al. (1996) J. Neuroimmunol. 69: 103-115.
Ito, A. et al. (2001) J. Immunol. 167(1): 542-52.
Jeffery, N. D. et al. (1995) J. Neurocytol. 24: 775-781.
Johns, T. G. et al. (1995) J. Immunol. 154: 5536-3341.
Karpus, W. J. et al. (1995) J. Immunol. 155(10): 5003-10.
Kawai, K. et al. (1996) Cell Immunol. 171(2): 262-8.
Kennedy, B. P. et al. (1995) J. Biol. Chem. 270: 22378-22385.
Kent, S. J. et al. (1995) J. Neuroimmunol. 58(1): 1-10.
Klivenyl, P. et al. (1998) J. Neurochem. 71: 2634-2637.
Kohler, G. et al. (1975) Nature 256: 495-497.
Komada, M. et al. (1989) J. Biochem. 106: 545-547.
Kramer, R. M. et al. (1989) J. Biol. Chem. 264: 5768-5775.
Lautens, L. L. et al. (1998) Brain Res. 809: 18-30.
Lee, S. J. et al. (1999) J. Neuroimmunol. 98: 77-88.
Leonard, J. P. et al. (1996) Ann. N.Y. Acad. Sci. 795: 216-26.
Lin, L. L. et al. (1992) J. Biol. Chem. 267: 23451-23454.
Lin, L. L. et al. (1993) Cell 72: 269-278.
Liu, J. et al. (1998) Nature Medicine 4: 78-8326.
Lyons-Giordano, B. et al. (1989) Biochem. Biophys. Res. Commun. 164: 488-495.
Martin, R. et al. (1990) J. Immunol. 145: 540-548.
Meyer, A. L. et al. (1996) J. Immunol. 157(9): 4230-8.
Mokhtarian, F. et al. (1984) Nature 309: 356-358.
Molloy, G. Y. et al. (1998) Neurosci. Lett. 258: 139-142.
Murakami, M. et al. (1990) Biochem. Biophys. Acta. 1043: 34-42.
Murakami, M. et al. (1993) J. Biol. Chem. 268: 839-844
Murakami, M. et al. (1997) Critical Reviews in Immunol. 17: 225-283.
Nalefski, E. A. et al. (1994) J. Biol. Chem. 269: 1823-1849.
Nakano, T. et al. (1990) F.E.B.S. Let. 273: 23-26.
Nakano, T. et al. (1990) F.E.B.S. Lett. 261: 171-174.
Norton, W. T. et al. (1990) Acta Neuropathologiae Experimentalis 50: 225-235.
Noseworthy, J. H. et al. (2000) N. Engl. J. Med. 28; 343(13): 938-52.
Ogata, K. et al. (2001) Transplantation 71: 1040-1046.
Oger, J. et al. (1994) Ann. Neurol. 36: S22-24.
Oka, S. et al. (1991) J. Biol. Chem. 266: 9956-9960.
Olsson, T. et al. (1990) J. Clin. Invest. 86: 981-985.
Oinuma et al. (1991) J. Med. Chem. 34: 2260-7.
Ousman, S. et al. (2000) Glia 30: 92-104.
Ousman, S. et al. (2001) J. Neurosci. 21: 4649-4656.
Owada, Y. et al. (1994) Mol. Brain Res. 25: 364-368.
Ozawa, K. et al. (1994) Brain 117: 1311-1322.
Panitch, H. S. et al. (1987) Neurology 37: 1097-1102.
Penkowa, M. et al. (2000) Glia. 32(3): 247-63.
Pitt, D. et al. (2000) Nat. Med. Jan. 6(1): 67-70.
Polman, C. H. et al. (2000) West J. Med. 173(6): 398-402.
Popovich, P. G. et al. (1997) J. Neuropathol. Exp. Neurol. 56(12): 1323-38.
Prineas, J. W. et al. (1993) Ann. Neurol. 33: 137-151.
Probert, L. et al. (1995) Proc. Natl. Acad. Sci. U.S.A. 92: 11294-11298.
Prokazova, N. V. et al. (1998) Biochemistry 63: 31-37.
Prosiegel, M. et al. (1989) Acta. Neurol. Scand. 79(3): 223-6.
Prosiegel, M. et al. (1990) Acta. Neurol. Scand. 81(3): 237-8.
Racke, M. K. et al. (1995) J. Immunol. 154(1): 450-8.
Raine, C. S. et al. (1992) Semin. Neurosci. 4: 201-211.
Reder, A. T. et al. (1994) J. Neuroimmunol. 54: 117-127.
Reichert, J. R. et al. (1989) Ann. Neurol. 26: 342-346.
Renno, T. et al. (1994) J. Neuroimmunol. 49: 1-7.
Reynolds et al. (1994) Anal. Biochem. 217: 25-32.
Rolak, L. A. (2001) Neurol. Clin. 19(1): 107-18.
Ross, B. M. et al. (1995) J. Neurochem. 64: 2213-2221.
Ruddle, N. H. et al. (1990) J. Exp. Med. 1990 172(4): 1193-200.
Ruuls et al. (1996) J. Immunol. 157:5721-5731.
Ryborg, A. K. et al. (1994) Arch. Dermatol. Res. 286: 462-465.
Sadovnick, A. D. et al. (1996) The Canadian Collaborative Study Group. Lancet 347: 1728-1730.
Seilhamer, J. J. et al. (1989) J. Biol. Chem. 264: 5335-5338.
Selmaj, K. et al. (1998) Eur. J. Immunol. 28(6): 2035-44.
Sharp, J. D. et al. (1991) J. Biol. Chem. 266: 14850-14853.
Smith, K. J. et al. (1999) Brain Pathol. 9: 69-92.
Smith, T. et al. (2000) Nat. Med. 6(1): 62-6.
Sobel, R. A. et al. (1990) Am. J. Pathol. 136: 1309-1316.
Steinman, L. (1999) Neuron. 24: 511-4.
Steinman, L. et al. (1994) Annu. Rev. Neurosci. 17: 247-65.
Stinissen, P. et al. (1997) Crit. Rev. Immunol. 17(1): 33-75.
Street et al. (1993) Biochemistry 32: 5935-40.
Sun, J. B. et al. (1991) Eur. J. Immunol. 21: 1461-1468.
Taupin, V. et al. (1997) Eur. J. Immunol. 27: 905-913.
Tienari, P. (1994) Annals of Med. 26: 259-269.
Tran, E. H. et al. (1998) J. Immunol. 161(7): 3767-75.
Trapp, B. D. et al. (1998) N. Engl. J. Med. 338: 278-85.
Traugott, U. et al. (1983) Science 219: 308-310.
Trotter, J. et al. (1986) Neurochem. Res. 11: 349-361.
Tuohy, V. K. (1994) Neurochemical Res. 19: 935-944.
van der Meide et al. (1998) J. Neuroimmunol. 84: 14-23.
Warren, K. G. et al. (1994) Ann. Neurol. 35: 280-289.
Weber, F. et al. (1989) Int. Arch. Allergy Appl. Immunol. 89(2-3): 242-5.
Woelk, H. et al. (1974) J. Neurol. 207: 319-326.
Wright, G. W. et al. (1989) J. Biol. Chem. 265: 6675-6681.
Wu, T. et al. (1994) J. Clin. Invest. 93: 571-577.
Wujek, J. R. et al. (2002) J. Neuropathol. Exp. Neurol. 61: 23-32.
Xu, X. X. et al. (1994) J. Biological Chem. 269: 31693-31700.
Yang, H. C. et al. (1994) Biochem. J. 299: 91.
Yelton, D. E. et al. (1981) Ann. Rev. Biochem. 50: 657-680.
Yednock, T. A. et al. (1992) Nature 356(6364): 63-6.
Yu, M. et al. (1996) J. Neuroimmunol. 64(1): 91-100.
Zamvil, S. et al. (1985) Nature 317: 355-358.
Zhang, Y. et al. (1993) Neurology 43: 403-407.

1. A method of preventing or treating a neural inflammatory or demyelinating disease in an animal, said method comprising inhibiting the activity of a phospholipase A₂ in the animal.

2. The method of claim 1, wherein the animal is a mammal.

3. The method of claim 1, wherein the animal is a human.

4. The method of claim 1, wherein the neural inflammatory or demyelinating disease is selected from the group consisting of Multiple Sclerosis, Alzheimer's disease, amyotrophic lateral sclerosis and stroke.

5. The method of claim 1, wherein the phospholipase A₂ is a cytosolic phospholipase A₂.

6. The method of claim 1, wherein the method comprises administering to the animal an effective amount of a phospholipase A₂ inhibitor.

7. The method of claim 1, wherein the inhibitor is selected from the group consisting of arachidonic acid analogues, benzenesulfonamide derivatives, bromoenol lactone, p-bromophenyl bromide, bromophenacyl bromide, trifluoromethylketones, sialoglycolipids and proteoglycans.

8. The method of claim 7, wherein the inhibitor is selected from the group consisting of arachidonyl trifluoromethyl ketone, methyl arachidonyl fluorophosphonate, palmitoyl trifluoromethyl ketone.

9. The method of claim 1, wherein the method comprises inhibiting the expression of a phospholipase A₂.

10. The method of claim 9, wherein the method comprises administering to the animal an effective amount of a phospholipase A₂ inhibitor.

11. The method of claim 10 wherein said phospholipase A₂ inhibitor is selected from the group consisting of an antisense molecule and an siRNA or siRNA-like molecule.

12. The method of claim 11 wherein the antisense molecule is a nucleic acid that is substantially complementary to a portion of an mRNA encoding a phospholipase A₂.

13. The method of claim 12 wherein the antisense molecule is complementary to a portion of a nucleic acid sequence substantially identical to a sequence selected from the group consisting of SEQ ID NO:1 and SEQ ID NO:3.

14. The method of claim 12 wherein the portion of an mRNA comprises at least 5 contiguous bases.

15. The method of claim 10 wherein the siRNA or siRNA-like molecule is substantially identical to a portion of an mRNA encoding a phospholipase A₂.

16. The method of claim 10 wherein the siRNA or siRNA-like molecule is substantially identical to a portion of an mRNA corresponding to a DNA sequence selected from the group consisting of SEQ ID NO:1 and SEQ ID NO:3.

17. The method of claim 10 wherein the siRNA or siRNA-like molecule comprises less than about 30 nucleotides.

18. The method of claim 17 wherein the siRNA or siRNA-like molecule comprises about 21 to about 23 nucleotides.

19-35. (canceled)

36. A commercial package comprising a phospholipase A₂ inhibitor together with instructions for preventing or treating a neural inflammatory or demyelinating disease in an animal.

37. The commercial package of claim 36, wherein the animal is a mammal.

38. The commercial package of claim 37, wherein the mammal is a human.

39. The commercial package of claim 36, wherein the neural inflammatory or demyelinating disease is selected from the group consisting of Multiple Sclerosis, Alzheimer's disease, amyotrophic lateral sclerosis and stroke.

40. The commercial package of claim 36, wherein the phospholipase A₂ is a cytosolic phospholipase A₂.

41. The commercial package of claim 36, wherein the inhibitor is selected from the group consisting of arachidonic acid analogues, benzenesulfonamide derivatives, bromoenol lactone, p-bromophenyl bromide, bromophenacyl bromide, trifluoromethylketones, sialoglycolipids and proteoglycans.

42. The commercial package of claim 36, wherein the inhibitor is selected from the group consisting of arachidonyl trifluoromethyl ketone, methyl arachidonyl fluorophosphonate, palmitoyl trifluoromethyl ketone.

43. The commercial package of claim 36, wherein the inhibitor is an inhibitor of phospholipase A₂ expression.

44. The commercial package of claim 43 wherein said phospholipase A₂ inhibitor is selected from the group consisting of an anti sense molecule and an siRNA or siRNA-like molecule.

45. The commercial package of claim 44 wherein the antisense molecule is a nucleic acid that is substantially complementary to a portion of an mRNA encoding a phospholipase A₂.

46. The commercial package of claim 44 wherein the antisense molecule is complementary to a portion of a nucleic acid sequence substantially identical to a sequence selected from the group consisting of SEQ ID NO:1 and SEQ ID NO:3.

47. The commercial package of claim 45 wherein the portion of an mRNA comprises at least 5 contiguous bases.

48. The commercial package of claim 44 wherein the siRNA or siRNA-like molecule is substantially identical to a portion of an mRNA encoding a phospholipase A₂.

49. The commercial package of claim 44 wherein the siRNA or siRNA-like molecule is substantially identical to a portion of an mRNA corresponding to a DNA sequence selected from the group consisting of SEQ ID NO:1 and SEQ ID NO:3.

50. The commercial package of claim 44 wherein the siRNA or siRNA-like molecule comprises less than about 30 nucleotides.

51. The commercial package of claim 50 wherein the siRNA or siRNA-like molecule comprises about 21 to about 23 nucleotides.

52. A method of identifying a compound for prevention and/or treatment of neural inflammatory or demyelinating disease, said method comprising:
(a) providing a test compound; and
(b) determining whether activity or expression of a phospholipase A₂ is decreased in the presence of said test compound;
wherein a decrease in said activity is indicative that said test compound may be used for treating a neural inflammatory or demyelinating disease.

53. The method of claim 52, further comprising the step of assaying the compounds for activity in the prevention or treatment of a neural inflammatory or demyelinating disease.

54. The method of method of claim 52, wherein the phospholipase A₂ is a mammalian phospholipase A₂.

55. The method of claim 54, wherein the phospholipase A₂ is a human phospholipase A₂.

56. The method of claim 52, wherein the phospholipase A₂ is a cytosolic phospholipase A₂.

57. The method of claim 52, wherein the neural inflammatory or demyelinating disease is selected from the group consisting of Multiple Sclerosis, Alzheimer's disease, amyotrophic lateral sclerosis and stroke.

58-72. (canceled)

73. A method of identifying or characterizing a compound for prevention or treatment of a neural inflammatory or demyelinating disease, said method comprising:
(a) contacting a test compound with a cell comprising a first nucleic acid comprising a transcriptionally regulatory element normally associated with a PLA₂ gene, operably linked to a second nucleic acid comprising a reporter gene capable of encoding a reporter protein; and
(b) determining whether reporter gene expression or reporter protein activity is decreased in the presence of said test compound; said decrease in reporter gene expression or reporter protein activity being an indication that said test compound may be used for prevention or treatment of neural inflammatory or demyelinating disease.
